SEQUENCE LISTING (34 sequences)

SEQ ID NO: 1 - DNA, 1422 bp, Zymomonas mobilis
atgagttctgaaagtagtcagggtctagtcacgcgactagccctaatcgctgctataggcggcttgcttttcggttacgattcagcggttatcgctgcaatcggtacaccggttgatatccattttattgcccctcgtcacctgtctgctacggctgcggcttccctttctgggatggtcgttgttgctgttttggtcggttgtgttaccggttctttgctgtctggctggattggtattcgcttcggtcgtcgcggcggattgttgatgagttccatttgtttcgtcgccgccggttttggtgctgcgttaaccgaaaaattatttggaaccggtggttcggctttacaaattttttgctttttccggtttcttgccggtttaggtatcggtgtcgtttcaaccttgaccccaacctatattgctgaaattgctccgccagacaaacgtggtcagatggtttctggtcagcagatggccattgtgacgggtgctttaaccggttatatctttacctggttactggctcatttcggttctatcgattgggttaatgccagtggttggtgctggtctccggcttcagaaggcctgatcggtattgccttcttattgctgctgttaaccgcaccggatacgccgcattggttggtgatgaagggacgtcattccgaggctagcaaaatccttgctcgtctggaaccgcaagccgatcctaatctgacgattcaaaagattaaagctggctttgataaagccatggacaaaagcagcgcaggtttgtttgcttttggtatcaccgttgtttttgccggtgtatccgttgctgccttccagcagttagtcggtattaacgccgtgctgtattatgcaccgcagatgttccagaatttaggttttggagctgatacggcattattgcagaccatctctatcggtgttgtgaacttcatcttcaccatgattgcttcccgtgttgttgaccgcttcggccgtaaacctctgcttatttggggtgctctcggtatggctgcaatgatggctgttttaggctgctgtttctggttcaaagtcggtggtgttttgcctttggcttctgtgcttctttatattgcagtctttggtatgtcatggggccctgtctgctgggttgttctgtcagaaatgttcccgagttccatcaagggcgcagctatgcctatcgctgttaccggacaatggttagctaatatcttggttaacttcctgtttaaggttgccgatggttctccagcattgaatcagactttcaaccacggtttctcctatctcgttttcgcagcattaagtatcttaggtggcttgattgttgctcgcttcgtgccggaaaccaaaggtcggagcctggatgaaatcgaggagatgtggcgctcccagaagtag

SEQ ID NO: 2 - DNA, 975 bp, Zymomonas mobilis
atggaaattgttgcgattgacatcggtggaacgcatgcgcgtttctctattgcggaagtaagcaatggtcgggttctttctcttggagaagaaacaacttttaaaacggcagaacatgctagcttgcagttagcttgggaacgtttcggtgaaaaactgggtcgtcctctgccacgtgccgcagctattgcatgggctggcccggttcatggtgaagttttaaaacttaccaataacccttgggtattaagaccagctactctgaatgaaaagctggacatcgatacgcatgttctgatcaatgacttcggcgcggttgcccacgcggttgcgcatatggattcttcttatctggatcatatttgtggtcctgatgaagcgcttcctagcgatggtgttatcactattcttggtccgggaacgggcttgggtgttgcccatctgttgcggactgaaggccgttatttcgtcatcgaaactgaaggcggtcatatcgactttgctccgcttgacagacttgaagacaaaattctggcacgtttacgtgaacgtttccgccgcgtttctatcgaacgcattatttctggcccgggtcttggtaatatctacgaagcactggctgccattgaaggcgttccgttcagcttgctggatgatattaaattatggcagatggctttggaaggtaaagacaaccttgctgaagccgctttggatcgcttctgcttgagccttggcgctatcgctggtgatcttgctttggcacagggtgcaaccagtgttgttattggcggtggtgtcggtcttcgtatcgcttcccatttgccagaatctggtttccgtcagcgctttgtttcaaaaggacgctttgaacgcgtcatgtccaagattccggttaagttgattacttatccgcagcctggactgttgggtgcggcagctgcctatgccaacaaatattctgaagttgaataa

SEQ ID NO: 3 - DNA, 1248 bp, Escherichia coli
atggcactgaatattccattcagaaatgcgtactatcgttttgcatccagttactcatttctcttttttatttcctggtcgctgtggtggtcgttatacgctatttggctgaaaggacatctagggttgacagggacggaattaggtacactttattcggtcaaccagtttaccagcattctatttatgatgttctacggcatcgttcaggataaactcggtctgaagaaaccgctcatctggtgtatgagtttcatcctggtcttgaccggaccgtttatgatttacgtttatgaaccgttactgcaaagcaatttttctgtaggtctaattctgggggcgctattttttggcttggggtatctggcgggatgcggtttgcttgatagcttcaccgaaaaaatggcgcgaaattttcatttcgaatatggaacagcgcgcgcctggggatcttttggctatgctattggcgcgttctttgccggcatattttttagtatcagtccccatatcaacttctggttggtctcgctatttggcgctgtatttatgatgatcaacatgcgttttaaagataaggatcaccagtgcgtagcggcagatgcgggaggggtaaaaaaagaggattttatcgcagttttcaaggatcgaaacttctgggttttcgtcatatttattgtggggacgtggtctttctataacatttttgatcaacaactttttcctgtcttttattcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggagttgtgattatggcgttgcgtatcctttcctgcgcgctgttcgttaacccctggattatttcattagtgaagttgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcttgagtaaaaaacgcgagcaaatagttatggaaacgcctgtaccttcagcaatatag

SEQ ID NO: 4 - DNA, 1437 bp, Escherichia coli
atgttaacccagtagccagagtgctccatgttgcagcacagccactccgtgggaggcataaagcgacagttcccgttcttctggctgcggatagattcgactactcatcaccgcttccccgtcgttaataaatacttccacggatgatgtatcgataaatatccttagggcgagcgtgtcacgctgcgggaggggaatactacggtagccgtctaaattctcgtgtgggtaataccgccacaaaacaagtcgctcagattggttatcaatatacagccgcattccagtgccgagctgtaatccgtaatgttcggcatcactgttcttcagcgcccactgcaactgaatctcaactgcttgcgcgttttcctgcaaaacatatttattgctgattgtgcggggagagacagattgatgctgctggcgtaacgactcagcttcgtgtaccgggcgttgtagaagtttgccattgctctctgatagctcgcgcgccagcgtcatgcagcctgcccatccttcacgttttgagggcattggcgattcccacatatccatccagccgataacaatacgccgaccatccttcgctaaaaagctttgtggtgcataaaagtcatgcccgttatcaagttcagtaaaatgcccggattgtgcaaaaagtcgtcctggcgaccacattccgggtattacgccactttgaaagcgatttcggtaactgtatccctcggcattcattccctgcggggaaaacatcagataatgctgatcgccaaggctgaaaaagtccggacattcccacatatagctttcacccgcatcagcgtgggccagtacgcgatcgaaggtccattcacgcaacgaactgccgcgataaagcaggatctgccccgtgttgcctggatctttcgccccgactaccatccaccatgtgtcggcttcacgccacactttaggatcgcggaagtgcatgattccttctggtggagtgaggatcacaccctgtttctcgaaatgaataccatcccgactggtagccagacattgtacttcgcgaattgcatcgtcattacctgcaccatcgagccagacgtgtccggtgtagataagtgagaggacaccattgtcatcgacagcactacctgaaaaacacccgtctttgtcattatcgtctcctggcgctagcgcaataggctcatgctgccagtggatcatatcgtcgctggtggcatgtccccagtgcattggcccccagtgttcgctcatcggatgatgttgataaaacgcgtgataacgatcgttaaaccagatcaggccgtttggatcgttcatccacccggcaggaggcgcgaggtgaaaatggggatagaaagtgttaccccggtgctcatgaagttttgctagggcgttttgcgccgcatgcaatcgagattgcgtcat

SEQ ID NO: 5 - DNA, 915 bp, Escherichia coli
atgtcagccaaagtatgggttttaggggatgcggtcgtagatctcttgccagaatcagacgggcgcctactgccttgtcctggcggcgcgccagctaacgttgcggtgggaatcgccagattaggcggaacaagtgggtttataggtcgggtgggggatgatccttttggtgcgttaatgcaaagaacgctgctaactgagggagtcgatatcacgtatctgaagcaagatgaatggcaccggacatccacggtgcttgtcgatctgaacgatcaaggggaacgttcatttacgtttatggtccgccccagtgccgatctttttttagagacgacagacttgccctgctggcgacatggcgaatggttacatctctgttcaattgcgttgtctgccgagccttcgcgtaccagcgcatttactgcgatgacggcgatccggcatgccggaggttttgtcagcttcgatcctaatattcgtgaagatctatggcaagacgagcatttgctccgcttgtgtttgcggcaggcgctacaactggcggatgtcgtcaagctctcggaagaagaatggcgacttatcagtggaaaaacacagaacgatcaggatatatgcgccctggcaaaagagtatgagatcgccatgctgttggtgactaaaggtgcagaaggggtggtggtctgttatcgaggacaagttcaccattttgctggaatgtctgtgaattgtgtcgatagcacgggggcgggagatgcgttcgttgccgggttactcacaggtctgtcctctacgggattatctacagatgagagagaaatgcgacgaattatcgatctcgctcaacgttgcggagcgcttgcagtaacggcgaaaggggcaatgacagcgctgccatgtcgacaagaactggaatag

SEQ ID NO: 6 - DNA, 768 bp, Escherichia coli
atgcgacatcctttagtgatgggtaactggaaactgaacggcagccgccacatggttcacgagctggtttctaacctgcgtaaagagctggcaggtgttgctggctgtgcggttgcaatcgcaccaccggaaatgtacatcgatatggcgaagcgcgaagctgaaggcagccacatcatgctgggtgcgcaaaacgtggacctgaacctgtccggcgcattcaccggtgaaacttctgccgctatgctgaaagacatcggcgctcagtacatcatcatcggccactctgaacgtcgtacttaccacaaagagtctgacgaactgatcgcgaaaaaattcgcggtgctgaaagagcagggcctgactccggttctgtgcatcggtgaaaccgaagctgaaaacgaagcgggcaaaactgaagaagtttgcgcacgtcagatcgacgcggtactgaaaactcagggtgctgcggcattcgaaggtgcggttatcgcttacgaacctgtatgggcaatcggtactggcaaatctgcaactccggctcaggcacaggctgttcacaaattcatccgtgaccacatcgctaaagttgacgctaacatcgctgaacaagtgatcattcagtacggcggctctgtaaacgcgtctaacgctgcagaactgtttgctcagccagatatcgacggcgcgctggttggcggtgcttctctgaaagctgacgctttcgcagtgatcgttaaagctgcagaagcggctaaacaggcttaa

SEQ ID NO: 7 - DNA, 500 bp, Escherichia coli
cggggaaaaacaaacgttattacaccgagacagaaggtgcactgcgttatgttgtcgcggacaacggcgaaaaggggctgaccttcgctgttgaaccgattaagctggcgctatctgaatcgcttgaaggtttgaataaatgacaaaaagcaaagcctttgtgccgatgaatctctatactgtttcacagacctgctgccctgcggggcggccatcttcctttattcgcttataagcgtggagaattaaagtctgacaggtgccggatttcatatccggcacttactttccttaactcttcgccttaacgcaaaatctcacactgatgatcctgaatttcctcggctgaagcacggttaagcgtcagtagatttcgttgtgtcgccagcaatacaaatgagttatcactctgccgtaccatcgccagcccgtagcttcccatatgttcccgcgcctcaggtacttcttctgccagcattataaatgggctgcgttgtacc

SEQ ID NO: 8 - DNA, 3568 bp, Escherichia coli
atgatttatcctgatgaagcaatgctttacgcaccggttgaatggcacgactgctccgaaggtttcgaggacattcgttatgaaaaatccaccgacggtatcgcaaaaatcaccattaatcgtccgcaggtgcgcaatgccttccgtcctctgacggtaaaagagatgatccaggcgctggcagatgcgcgttatgacgacaacatcggcgtgatcattctgactggtgcaggcgataaagcgttctgctccggtggtgaccagaaagtgcgtggtgattacggcggctataaagatgattccggcgtacatcacctgaatgtgctggacttccagcgtcagatccgtacctgtccgaaaccggttgtcgcgatggtggctggctactccatcggcggcggtcacgttctgcacatgatgtgcgacctgactatcgcggcagataatgccatcttcggtcagactggcccgaaagtcggttccttcgacggcggctggggcgcttcctacatggctcgcatcgtcgggcagaaaaaagcgcgtgaaatctggttcctgtgccgtcagtacgacgcaaaacaggcgctggatatgggccttgtgaacaccgtggtaccgctggcggatctggaaaaagaaaccgtccgttggtgccgcgaaatgctgcaaaacagcccgatggcgctgcgctgcctgaaagctgcactgaacgccgactgtgacgggcaggcggggctgcaggagctggcgggcaacgccaccatgctgttctacatgacggaagaaggtcaggaaggtcgcaacgccttcaaccagaaacgtcagcctgacttcagcaaattcaaacggaatccgtaatgcgtagcgcgcaggtataccgctggcagatccccatggacgcgggggtggttctgcgcgacaggcggttaaaaacccgcgacgggctgtatgtttgcctgcgtgaaggcgagcgcgaagggtggggggagatctcccataagcgctaacttaagggttgtggtattacgcctgatatgatttaacgtgccgatgaattactctcacgataactggtcagcaattctggcccatattggtaagcccgaagaactggatacttcggcacgtaatgccggggctctaacccgccgccgcgaaattcgtgatgctgcaactctgctacgtctggggctggcttacggccccggggggatgtcattacgtgaagtcactgcatgggctcagctccatgacgttgcaacattatctgacgtggctctcctgaagcggctgcggaatgccgccgactggtttggcatacttgccgcacaaacacttgctgtacgcgccgcagttacgggttgtacaagcggaaagagattgcgtcttgtcgatggaacagcaatcagtgcgcccgggggcggcagcgctgaatggcgactacatatgggatatgatcctcatacctgtcagttcactgattttgagctaaccgacagcagagacgctgaacggctggaccgatttgcgcaaacggcagacgagatacgcattgctgaccggggattcggttcgcgtcccgaatgtatccgctcacttgcttttggagaagctgattatatcgtccgggttcactggcgaggattgcgctggttaactgcagaaggaatgcgctttgacatgatgggttttctgcgcgggctggattgcggtaagaacggtgaaaccactgtaatgataggcaattcaggtaataaaaaagccggagctccctttccggcacgtctcattgccgtatcacttcctcccgaaaaagcattaatcagtaaaacccgactgctcagcgagaatcgtcgaaaaggacgagtagttcaggcggaaacgctggaagcagcgggccatgtgctattgctaacatcattaccggaagatgaatattcagcagagcaagtggctgattgttaccgtctgcgatggcaaattgaactggcttttaagcggctcaaaagtttgctgcacctggatgctttgcgtgcaaaggaacctgaactcgcgaaagcgtggatatttgctaatctactcgccgcatttttaattgacgacataatccagccatcgctggatttcccccccagaagtgccggatccgaaaagaagaactaactcgttgtggagaataacaaaaatggtcatctggagcttacaggtggccattcgtgggacagtatccctgacagcctacaaaacgcaattgaagaacgcgaggcatcgtcttaacgaggcaccgaggcgtcgcattcttcagatggttcaacccttaagttagcgcttatgggaattatccccggcttttttatgtatggtcttacagcaccagtgctgcgattgacgcagacagcacactcaccagggtagagccgtaaaccagcttcagaccgaagcgagaaaccacgttaccttgctcttcattcaggcctttaactgcacctgcgataatcccgattgaagagaagttagcgaaggaaaccaggaacacagagatgatgccttcagcacgcggagagagcgtggaagcaattttctgcagatccatcatcgcaacgaactcgttggaaaccagtttggtcgccatgatactgcccacttgcagtgcttcactggaaggaacacccatcacccatgcaatcggatagaagatgtagcccaggatgccctggaaggagatgctgtagccaaaccagccagtaacggtggcaaacagtgcgttcagcgcggcgatcagggcgataaagccaatcagcatcgcggcaacgataatggcaactttgaaacctgccagaatgtattcacccagcatttcgaagaagctctgaccttcgtgcaggttggacatctggatgttttcttcactggcatcaacacggtaaggattgatcagcgacagcacgataaaggtgctgaacatgttcagtaccagcgcagcaacgacgtatttcggttccagcatggtcatgtatgcaccaacgatggacatcgacacggtggacattgccgtggcagccatggtgtacatacgattacgggagattttgccgaggatatctttataggcaataaagttttcagactgacccagaatcagggagctgacggcgttaaaggattccagtttgcccatgccgttgactttggagagcaggaaaccaattgcgcggatgatcaccggcaacacgcgaatgtgctggagaataccgatcagtgcagagataaagacgattgggcacagcactttcaggaagaagaatgccaggccttgatcattcatgctaccaaagacgaagttagtcccttcgttggcaaatccgagcagtttttcgaacatttcggagaagcctttcacgaagcctaaaccaacgtcggagttcaggaagaaccacgccagtaacacttcgataacaagcagttgaataacataacggatacgaatttttttgcggtcgctgcttaccagcagtgcgagaatcgcaacaacggcaagtgccagtacaaaatgaaggacgcggtccat

SEQ ID NO: 9 - DNA, 1600 bp, Escherichia coli
caacgccttcaaccagaaacgtcagcctgacttcagcaaattcaaacggaatccgtaatgcgtagcgcgcaggtataccgctggcagatccccatggacgcgggggtggttctgcgcgacaggcggttaaaaacccgcgacgggctgtatgtttgcctgcgtgaaggcgagcgcgaagggtggggggagatctcccataagcgctaacttaagggttgtggaaaaggggctgaccttcgctgttgaaccgattaagctggcgctatctgaatcgcttgaaggtttgaataaatgacaaaaagcaaagcctttgtgccgatgaatctctatactgtttcacagacctgctgccctgcggggcggccatcttcctttattcgcttataagcgtggagaattaaaatgcgacatcctttagtgatgggtaactggaaactgaacggcagccgccacatggttcacgagctggtttctaacctgcgtaaagagctggcaggtgttgctggctgtgcggttgcaatcgcaccaccggaaatgtacatcgatatggcgaagcgcgaagctgaaggcagccacatcatgctgggtgcgcaaaacgtggacctgaacctgtccggcgcattcaccggtgaaacttctgccgctatgctgaaagacatcggcgctcagtacatcatcatcggccactctgaacgtcgtacttaccacaaagagtctgacgaactgatcgcgaaaaaattcgcggtgctgaaagagcagggcctgactccggttctgtgcatcggtgaaaccgaagctgaaaacgaagcgggcaaaactgaagaagtttgcgcacgtcagatcgacgcggtactgaaaactcagggtgctgcggcattcgaaggtgcggttatcgcttacgaacctgtatgggcaatcggtactggcaaatctgcaactccggctcaggcacaggctgttcacaaattcatccgtgaccacatcgctaaagttgacgctaacatcgctgaacaagtgatcattcagtacggcggctctgtaaacgcgtctaacgctgcagaactgtttgctcagccagatatcgacggcgcgctggttggcggtgcttctctgaaagctgacgctttcgcagtgatcgttaaagctgcagaagcggctaaacaggcttaagtctgacaggtgccggatttcatatccggcacttactttccttaactcttcgccttaacgcaaaatctcacactgatgatcctgaatttcctcggctgaagcacggttaagcgtcagtagatttcgttgtgtcgccagcaatacaaatgagttatcactctgccgtaccatcgccagcccgtagcttcccattattacgcctgatatgatttaacgtgccgatgaattactctcacgataactggtcagcaattctggcccatattggtaagcccgaagaactggatacttcggcacgtaatgccggggctctaacccgccgccgcgaaattcgtgatgctgcaactctgctacgtctggggctggcttacggccccggggggatgtcattacgtgaagtcactgcatgggctcagctccatgacgttgc

SEQ ID NO: 10 - DNA, 4500 bp, Escherichia coli
caacgccttcaaccagaaacgtcagcctgacttcagcaaattcaaacggaatccgtaatgcgtagcgcgcaggtataccgctggcagatccccatggacgcgggggtggttctgcgcgacaggcggttaaaaacccgcgacgggctgtatgtttgcctgcgtgaaggcgagcgcgaagggtggggggagatctcccataagcgctaacttaagggttgtggaataactccctataatgcgccaccactgatccgttgttccacctgatattatgttaacccagtagccagagtgctccatgttgcagcacagccactccgtgggaggcataaagcgacagttcccgttcttctggctgcggatagattcgactactcatcaccgcttccccgtcgttaataaatacttccacggatgatgtatcgataaatatccttagggcgagcgtgtcacgctgcgggaggggaatactacggtagccgtctaaattctcgtgtgggtaataccgccacaaaacaagtcgctcagattggttatcaatatacagccgcattccagtgccgagctgtaatccgtaatgttcggcatcactgttcttcagcgcccactgcaactgaatctcaactgcttgcgcgttttcctgcaaaacatatttattgctgattgtgcggggagagacagattgatgctgctggcgtaacgactcagcttcgtgtaccgggcgttgtagaagtttgccattgctctctgatagctcgcgcgccagcgtcatgcagcctgcccatccttcacgttttgagggcattggcgattcccacatatccatccagccgataacaatacgccgaccatccttcgctaaaaagctttgtggtgcataaaagtcatgcccgttatcaagttcagtaaaatgcccggattgtgcaaaaagtcgtcctggcgaccacattccgggtattacgccactttgaaagcgatttcggtaactgtatccctcggcattcattccctgcggggaaaacatcagataatgctgatcgccaaggctgaaaaagtccggacattcccacatatagctttcacccgcatcagcgtgggccagtacgcgatcgaaggtccattcacgcaacgaactgccgcgataaagcaggatctgccccgtgttgcctggatctttcgccccgactaccatccaccatgtgtcggcttcacgccacactttaggatcgcggaagtgcatgattccttctggtggagtgaggatcacaccctgtttctcgaaatgaataccatcccgactggtagccagacattgtacttcgcgaattgcatcgtcattacctgcaccatcgagccagacgtgtccggtgtagataagtgagaggacaccattgtcatcgacagcactacctgaaaaacacccgtctttgtcattatcgtctcctggcgctagcgcaataggctcatgctgccagtggatcatatcgtcgctggtggcatgtccccagtgcattggcccccagtgttcgctcatcggatgatgttgataaaacgcgtgataacgatcgttaaaccagatcaggccgtttggatcgttcatccacccggcaggaggcgcgaggtgaaaatggggatagaaagtgttaccccggtgctcatgaagttttgctagggcgttttgcgccgcatgcaatcgagattgcgtcattttaatcatcctggttaagcaaatttggtgaattgttaacgttaacttttataaaaataaagtcccttactttcataaatgcgatgaatatcacaaatgttaacgttaactatgacgttttgtgatcgaatatgcatgttttagtaaatccatgacgattttgcgaaaaagaggtttatcactatgcgtaactcagatgaatttaagggaaaaaaatgtcagccaaagtatgggttttaggggatgcggtcgtagatctcttgccagaatcagacgggcgcctactgccttgtcctggcggcgcgccagctaacgttgcggtgggaatcgccagattaggcggaacaagtgggtttataggtcgggtgggggatgatccttttggtgcgttaatgcaaagaacgctgctaactgagggagtcgatatcacgtatctgaagcaagatgaatggcaccggacatccacggtgcttgtcgatctgaacgatcaaggggaacgttcatttacgtttatggtccgccccagtgccgatctttttttagagacgacagacttgccctgctggcgacatggcgaatggttacatctctgttcaattgcgttgtctgccgagccttcgcgtaccagcgcatttactgcgatgacggcgatccggcatgccggaggttttgtcagcttcgatcctaatattcgtgaagatctatggcaagacgagcatttgctccgcttgtgtttgcggcaggcgctacaactggcggatgtcgtcaagctctcggaagaagaatggcgacttatcagtggaaaaacacagaacgatcaggatatatgcgccctggcaaaagagtatgagatcgccatgctgttggtgactaaaggtgcagaaggggtggtggtctgttatcgaggacaagttcaccattttgctggaatgtctgtgaattgtgtcgatagcacgggggcgggagatgcgttcgttgccgggttactcacaggtctgtcctctacgggattatctacagatgagagagaaatgcgacgaattatcgatctcgctcaacgttgcggagcgcttgcagtaacggcgaaaggggcaatgacagcgctgccatgtcgacaagaactggaatagtgagaagtaaacggcgaagtcgctcttatctctaaataggacgtgaattttttaacgacaggcaggtaattatggcactgaatattccattcagaaatgcgtactatcgttttgcatccagttactcatttctcttttttatttcctggtcgctgtggtggtcgttatacgctatttggctgaaaggacatctagggttgacagggacggaattaggtacactttattcggtcaaccagtttaccagcattctatttatgatgttctacggcatcgttcaggataaactcggtctgaagaaaccgctcatctggtgtatgagtttcatcctggtcttgaccggaccgtttatgatttacgtttatgaaccgttactgcaaagcaatttttctgtaggtctaattctgggggcgctattttttggcttggggtatctggcgggatgcggtttgcttgatagcttcaccgaaaaaatggcgcgaaattttcatttcgaatatggaacagcgcgcgcctggggatcttttggctatgctattggcgcgttctttgccggcatattttttagtatcagtccccatatcaacttctggttggtctcgctatttggcgctgtatttatgatgatcaacatgcgttttaaagataaggatcaccagtgcgtagcggcagatgcgggaggggtaaaaaaagaggattttatcgcagttttcaaggatcgaaacttctgggttttcgtcatatttattgtggggacgtggtctttctataacatttttgatcaacaactttttcctgtcttttattcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggagttgtgattatggcgttgcgtatcctttcctgcgcgctgttcgttaacccctggattatttcattagtgaagttgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcttgagtaaaaaacgcgagcaaatagttatggaaacgcctgtaccttcagcaatatagacgtaaactttttccggttgttgtcgatagctctatatccctcaaccggaaaataataatagtaaaatgcttagccctgctaataatcgcctaatccaaacgcctgacacggaacaacggcaaacactattacgcctgatatgatttaacgtgccgatgaattactctcacgataactggtcagcaattctggcccatattggtaagcccgaagaactggatacttcggcacgtaatgccggggctctaacccgccgccgcgaaattcgtgatgctgcaactctgctacgtctggggctggcttacggccccggggggatgtcattacgtgaagtcactgcat

SEQ ID NO: 11 - DNA, 807 bp, Kluyveromyces marxianus
tatacctcaatcaaaactgaaattaggtgcctgtcacggctctttttttactgtacctgtgacttcctttcttatttccaaggatgctcatcacaatacgcttctagatctattatgcattataattaatagttgtagctacaaaaggtaaaagaaagtccggggcaggcaacaatagaaatcggcaaaaaaaactacagaaatactaagagcttcttccccattcagtcatcgcatttcgaaacaagaggggaatggctctggctagggaactaaccaccatcgcctgactctatgcactaaccacgtgactacatatatgtgatcgtttttaacatttttcaaaggctgtgtgtctggctgtttccattaattttcactgattaagcagtcatattgaatctgagctcatcaccaacaagaaatactaccgtaaaagtgtaaaagttcgtttaaatcatttgtaaactggaacagcaagaggaagtatcatcagctagccccataaactaatcaaaggaggatgtaagagttctccgagaacaagcagaggttcgagtgtactcggatcagaagttacaagttgatcgtttatatataaactatacagagatgttagagtgtaatggcattgcgcacattgtatacgctacaagtttagtcacgtgctagaagctgttttttgcaccgaaaatttttttttttttttttttttgttttttggtgaagtacattatgtgaaatttcacaaccaaagaaaaagagtttaatacaagtgcgaagaaccaaaccttgcttcttagtcca

SEQ ID NO: 12 - DNA, 4614 bp, Kluyveromyces marxianus
tagtttttgttcttccttcttttctgccaatgaagcactagtcatcctttatactgaaattcaattcagtttttgtagaatatgtcataccaatatcctatgactctttacaatatgtttatacatccattaatttaaccttaattgttattatcaatttttttttttcatttgacctaatctgtctttttggtaccttaataccctatggtgccttattcctgatatctgacatcaatctgaaaatttcaaaatcaagcattgctaagaagaacaaaaaaaaagtcacaagtaccgttcacagggtttggcaaaagacaagatagtatgccaatgccatgtaccgttcggtaagaacaaagtttgaactaagaaatgctcgaattataatcaggccgcctccccccattcgcttctctctgccatatgtcctttccgtcggccacgccctgggcccacgctccagcttccactttttggtgaacatttttttttttcccgggatccgaaactgcagcttcccaaaaaaccctgcagcacagcagcctccagcctccactaccaggccccacccagcttcccctatcccgccccccaaacaaactggccaaaaaaaaaataaaaaagcgtataaaacacaaaaaaaggcgtgatctcagcttccactatcattactggatgccccgctgcctacggctacggctacacccattggccattccatccatcaagccctcgggtataccataccacacaacctgattcagttcaaccgatccacactgtagtaacacacactatagactagcctataccataccataccataccatccatatacacacacatacacacacatacacacatacatacaccataccatacgacgcccatattcctctctctgcaaccggaggcagtttcgagtttcgagttgcgagtttatatgccttgactgacttcaagcagttgttggtttgactttctgaaatttttgttgctgcgctgtgctttcatgcgtttctttgattctgtgtatataagagtggacattgtaggtatatacgaatgaaacagagtgttgtttgatgaaaggttttccgatttcgttttagtatggattggattatttcataacccaaggacttacaaaggacttaagatacacatacacacatatataacctatcagacatggctagaacattcttcgttggtggtaacttcaagatgaacggtaccaaggcttccattaaggaaatcgttgagagattgaactctgcttcgattccatccaacgtggaagttgtgattgctcctccagctgcctacttggaccacgctgtctctttgaacaagaaggctcaagtcagtgttgctgcccaaaacgcatacttgaaggcttccggtgctttcactggtgaaaactctgtggagcaaatcaaggatgttggtgccgaatgggttatcttgggtcactccgaaagaagaacgtacttccacgaaaccgatgaattcattgctgacaagaccaagttcgctttggacagcggtgtcaaggttatcctatgtattggtgaaaccttggaagaaaagcaaaagggtatcactttggaagttgtccaaagacaattgcaagctgttttggacaaggtccaagactggaccaacgttgttgttgcctacgaaccagtctgggctattggtaccggtttggctgctacctctgacgatgctcaagacatccaccactccatcagagaattcttggccaagaagttgaacaaggacaccgctgaaaagatcagaatcctatacggtggttccgctaacggtaagaacgctgtcaccttcaaggacaaggccgacgttgacggtttcttggttggtggtgcttctttgaagccagaattcgttgacatcatcaactccagagtctaaattaaattaggttctagtccaaatacgaaatattaaaggaaaaaaaaacaataaataaataaagcctataaagctacgatgaaatagagagtgcttttgttttggaaaatttttgaaatgaatttaacggctgtatgagcacgcgcgataatgtagtgttgttactatatgatattgtatacttatatgtagcagtaagaacccgcttatcccaataacgaaataaaaacgaataacaataatttcaaatgtttatttgcattatttgaaactagggaagacaagcaacgaaacgtttttgaaaattttgagtattttcaataaatttgtagaggactcagatattgaaaaaaagctacagcaattaatacttgataagaagagtattgagaagggcaacggttcatcatctcatggatctgcacatgaacaaacaccagagtcaaacgacgttgaaattgaggctactgcgccaattgatgacaatacagacgatgataacaaaccgaagttatctgatgtagaaaaggattaaagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggcctttataaaaaggaactatccaatacctcgccagaaccaagtaacagtattttacggggcacaaatcaagaacaataagacaggactgtaaagtaacaataatttcaaatgtttatttgcattatttgaaactagggaagacaagcaacgaaacgtttttgaaaattttgagtattttcaataaatttgtagaggactcagatattgaaaaaaagctacagcaattaatacttgataagaagagtattgagaagggcaacggttcatcatctcatggatctgcacatgaacaaacaccagagtcaaacgacgttgaaattgaggctactgcgccaattgatgacaatacagacgatgataacaaaccgaagttatctgatgtagaaaaggattaattataaaagttttttttattgaaacttaaaacttaacgctactggtgtcaaatcatttttcatcatttttttctcggttactaagttagtctatcaaatcagtatcaataagaccgcatctggatcaaacaaggattatgtcaagaggaaattcaagtataatttacagcttgagttacgggatccggtaaagtccattggtgcgatgagaacgttgtattcactggtgctccggttattgtagactgaagtgaagcactaccgcatgagtaatgtctcttgtacagaaacgggttttaaggacgtatcgaccatagcaataaaagcaattatttgtttacattctatcaaaatgtgatttgttccagtcactacagatccttaaatcaacaaaacacatggcagcgccaacgcgacatatctaga

SEQ ID NO: 13 - DNA, 2422 bp, Kluyveromyces marxianus
atctgccgctgatgtttacgaccggtgttccggtttccaagagtccctattccgccatttcgagagaagcggcggcaagaagagagatgtggtcgcaattgtcacccacgggatattcttgcgtgtgttcttgatgaaatggtttagatggacttacgaggagtttgagtcgctcatgaacgtgccgaacggaagcgtcatcatcatggagctggacgaaacgctcgaccgctacgtgttgcgcaccaagctccccaaatggaccacaacggggtgcgatgatgccgcggcgggggcgtcaacctcgggctcgaactcttcttgcgcgcagaaacactgtcaatgcgagcccaccgcgatcgtctgatatcacgtgacccatacgtcattaccatatgtagcgatttaaacaaaaaaaaaagttgaaaaccgacagcagccgggtaacaattcacagcaactctctggctacagctctctggccacggctctctggcctcaggcctagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaagcttcatggcctttataaaaaggaactatccaatacctcgccagaaccaagtaacagtattttacggggcacaaatcaagaacaataagacaggactgtaaagaggcctgaacgagcaacattattgtttgattcccgacacaagatagcaatagcgaagatcattagttatttgttgaaacatgtatacgtatatatacataccatccatcatcacacgagaatgccgtaatacttggcccttggatacgctgggagtagatccggccattgcaccacaaactcgtctgccaatgggtacagaatcttcccagttagactcaacctgttggtccgcgccaacgactctacatacacaatcttggaaggctgccataagaacaaatggtacaatttcagccacacagtaattatacaacaggtcccgggcccattgagcaaagtgaggttcggtttacccctcatagacttcttgatattatacgtaatagtcatcgaagttatcagcgtctcgatgatacttatcacggaccctgcaaccgaactcccaacatcacgcgccttcttgaaatgatggtactctattttcacctgtagctgtttagaaga

SEQ ID NO: 14 - DNA, 21 bp, Artificial Sequence
  Primer sequence
aggaacgcttcggtgaactgg

SEQ ID NO: 15 - DNA, 21 bp, Artificial Sequence
  Primer sequence
tggtcttgcgacgttatgcgg

SEQ ID NO: 16 - DNA, 60 bp, Artificial Sequence
  Primer sequence
gactggcgctacaatcttccctgactccggttctgtgcatgttttagagctagaaatagc

SEQ ID NO: 17 - DNA, 500 bp, Artificial Sequence; identical to SEQ ID NO: 7
  Fragment sequence

SEQ ID NO: 18 - DNA, 21 bp, Artificial Sequence
  Primer sequence
acactcaccccattaatgacc

SEQ ID NO: 19 - DNA, 22 bp, Artificial Sequence
  Primer sequence
atctcttgtattcgtcctgatg

SEQ ID NO: 20 - DNA, 72 bp, Artificial Sequence
  Primer sequence
cgcgaagggtggggggagatctcccataagcgctaacttaagggttgtggaataactccctataatgcgcca

SEQ ID NO: 21 - DNA, 70 bp, Artificial Sequence
  Primer sequence
gttatcgtgagagtaattcatcggcacgttaaatcatatcaggcgtaatagtgtttgccgttgttccgtg

SEQ ID NO: 22 - DNA, 32 bp, Artificial Sequence
  Primer sequence
agggttgtggaataactccctataatgcgcca

SEQ ID NO: 23 - DNA, 30 bp, Artificial Sequence
  Primer sequence
aggcgtaatagtgtttgccgttgttccgtg

SEQ ID NO: 24 - DNA, 30 bp, Artificial Sequence
  Primer sequence
gggatatgatgtgagttatacacagggctg

SEQ ID NO: 25 - DNA, 30 bp, Artificial Sequence
  Primer sequence
catatccagctcagcttagtaaagccctcg

SEQ ID NO: 26 - DNA, 457 bp, Artificial Sequence
  Fragment sequence
actaagctgagctggatatgggccttgtgaacaccgtggtaccgctggcggatctggaaaaagaaaccgtccgttggtgccgcgaaatgctgcaaaacagcccgatggcgctgcgctgcctgaaagctgcactgaacgccgactgtgacgggcaggcggggctgcaggagctggcgggcaacgccaccatgctgttctacatgacggaagaaggtcaggaaggtcgcaacgccttcaaccagaaacgtcagcctgacttcagcaaattcaaacggaatccgtaatgcgtagcgcgcaggtataccgctggcagatccccatggacgcgggggtggttctgcgcgacaggcggttaaaaacccgcgacgggctgtatgtttgcctgcgtgaaggcgagcgcgaagggtggggggagatctcccataagcgctaacttaagggttgtggaataactccc

SEQ ID NO: 27 - DNA, 457 bp, Artificial Sequence
  Fragment sequence
cggcaaacactattacgcctgatatgatttaacgtgccgatgaattactctcacgataactggtcagcaattctggcccatattggtaagcccgaagaactggatacttcggcacgtaatgccggggctctaacccgccgccgcgaaattcgtgatgctgcaactctgctacgtctggggctggcttacggccccggggggatgtcattacgtgaagtcactgcatgggctcagctccatgacgttgcaacattatctgacgtggctctcctgaagcggctgcggaatgccgccgactggtttggcatacttgccgcacaaacacttgctgtacgcgccgcagttacgggttgtacaagcggaaagagattgcgtcttgtcgatggaacagcaatcagtgcgcccgggggcggcagcgctgaatggcgactacatatgggatatgatgtgagttata

SEQ ID NO: 28 - DNA, 20 bp, Artificial Sequence
  Primer sequence
gctggatatgggccttgtga

SEQ ID NO: 29 - DNA, 22 bp, Artificial Sequence
  Primer sequence
atcatatcccatatgtagtcgc

SEQ ID NO: 30 - DNA, 31 bp, Artificial Sequence
  Primer sequence
agggttgtggaaaaggggctgaccttcgctg

SEQ ID NO: 31 - DNA, 30 bp, Artificial Sequence
  Primer sequence
aggcgtaataatgggaagctacgggctggc

SEQ ID NO: 32 - DNA, 30 bp, Artificial Sequence
  Primer sequence
gactacatatggtacccagcttttgttccc

SEQ ID NO: 33 - DNA, 30 bp, Artificial Sequence
  Primer sequence
catatccagcggagctccaattcgccctat

SEQ ID NO: 34 - DNA, 457 bp, Artificial Sequence
  Fragment sequence
ttggagctccgctggatatgggccttgtgaacaccgtggtaccgctggcggatctggaaaaagaaaccgtccgttggtgccgcgaaatgctgcaaaacagcccgatggcgctgcgctgcctgaaagctgcactgaacgccgactgtgacgggcaggcggggctgcaggagctggcgggcaacgccaccatgctgttctacatgacggaagaaggtcaggaaggtcgcaacgccttcaaccagaaacgtcagcctgacttcagcaaattcaaacggaatccgtaatgcgtagcgcgcaggtataccgctggcagatccccatggacgcgggggtggttctgcgcgacaggcggttaaaaacccgcgacgggctgtatgtttgcctgcgtgaaggcgagcgcgaagggtggggggagatctcccataagcgctaacttaagggttgtggaaaaggggct

(19) United States
(12) Patent Application Publication
(10) Pub. No.: US 2020/0131538 A1
(43) Pub. Date: Apr. 30, 2020

(54) MICROORGANISM WITH STABILIZED COPY NUMBER OF FUNCTIONAL DNA SEQUENCE AND ASSOCIATED METHODS

(71) Applicant: PTT GLOBAL CHEMICAL PUBLIC COMPANY LIMITED, Bangkok (TH)

(72) Inventors: R. Rogers YOCUM, Lexington, MA (US); Tammy GRABAR, North Reading, MA (US); Theron HERMANN, Arlington, MA (US); Christopher Joseph MARTIN, Boston, MA (US); Ryan SILLERS, Reading, MA (US); Xiaohui YU, Newtown, MA (US); Xiaomei ZHOU, Belmont, MA (US)

(73) Assignee: PTT GLOBAL CHEMICAL PUBLIC COMPANY LIMITED, Bangkok (TH)

(21) Appl. No.: 16/626,805

(22) PCT Filed: Jun. 29, 2018
(86) PCT No.: PCT/US2018/040312

§ 371 Date: Dec. 26, 2019

Related U.S. Application Data
(60) Provisional application No. 62/584,270, filed on Nov. 10, 2017.
(60) Provisional application No. 62/527,442, filed on Jun. 30, 2017.

(51) Int. Cl. C12N 15/90; C12N 1/20; C12Q 1/04; C12Q 1/689

(52) U.S. Cl. CPC C12N 15/90; C12N 1/20; C12N 2510/00; C12Q 1/689; C12Q 1/04

(57) ABSTRACT

The invention provides processes for identifying and tracking genomic duplications that can occur during classical strain development or during the metabolic evolution of microbial strains originally constructed for the production of a biochemical through specific genetic manipulations, processes that stabilize the copy number of desirable genomic duplications using appropriate selectable markers, and non-naturally occurring microorganisms with stabilized copy numbers of a functional DNA sequence.

### CROSS-REFERENCE TO RELATED APPLICATIONS

This application claims priority to U.S. Provisional Patent Application No. 62/527,442 filed Jun. 30, 2017 and U.S. Provisional Patent Application No. 62/584,270 filed Nov. 10, 2017.

### STATEMENT REGARDING FEDERALLY SPONSORED RESEARCH OR DEVELOPMENT

Not Applicable.

### JOINT RESEARCH AGREEMENT

Not applicable.

### REFERENCE TO SEQUENCE LISTING

The Sequence Listing associated with this application is hereby incorporated by reference in its entirety. The text file containing the Sequence Listing was submitted electronically via the USPTO electronic filing system (EFS-Web).

### BACKGROUND OF INVENTION

The invention relates to the field of producing a chemical by fermentation of a microbe. More specifically, the invention relates to improving the economics of a process for producing a chemical by fermentation by increasing the titer of the desired chemical (also referred to as “fermentation product”). Even more specifically, the titer can be increased by creating, finding, and genetically stabilizing chromosomal DNA sequence duplications that increase the titer of the desired chemical. Once identified, a beneficial duplication can be combined with other beneficial genetic traits.

Microbes, such as eubacteria, yeasts, filamentous fungi, and archaea, can be genetically engineered and evolved to produce useful chemicals, for example organic acids, alcohols, polymers, amino acids, amines, carotenoids, fatty acids, esters, and proteins. In many cases, the production organism can be constructed by genetic engineering and/or classical genetics such that production of the desired chemical is coupled to growth, so that the only way the cells can grow is to metabolize the fed carbon source to the desired chemical (for example see Jantama et al, 2008a; Jantama et al, 2008b; (Zhu, Tan et al. 2014); U.S. Pat. Nos. 8,691,539; 8,871,489; WO Patent application WO2011063055A2). After such a strain has been constructed, it can be subjected to a process known as “metabolic evolution”, described as follows. For the method of metabolic evolution, a strain is grown under appropriate conditions, such as for the above example which involves production of succinic acid, in a minimal glucose medium under microaerobic conditions with pH control, for many generations, by allowing growth from a small inoculum (for example, at a low starting OD600 of about 0.01 to 0.5) for enough time for substantial growth to occur (for example, to an OD600 of about 1 to 30), and then inoculating that culture into fresh medium, again at a low OD600 of about 0.01 to 0.5, and then repeating the entire process many times until a faster growing strain evolves in the cultures. Each step of re-inoculation in the above example is also referred to as a “transfer”. The fermentations at each transfer can be batch or fed batch.

As an alternative, instead of repeated transfers into fresh medium, metabolic evolution can be accomplished in a continuous culture (also known as a “chemostat,” “auxostat,” or “pHstat”), where fresh medium is pumped or siphoned into a well-mixed fermentation vessel in a controlled fashion, and fermentation broth is removed at a similar rate, such that the working volume of the culture is held constant, and the rate of cell growth is able to keep up with the rate of dilution by the incoming fresh medium. In a chemostat, the feed rate is adjusted to maintain a particular cell density. In an auxostat, the feed rate is determined by measuring the concentration of a nutrient or product with the aim of maintaining a certain concentration of said nutrient or product. In a pHstat, the feed rate is adjusted to maintain a particular desired pH. As the rate of growth increases due to evolution, the rate of feeding is increased to allow continuous selection of faster growing variants.

During either type of metabolic evolution (i.e., transfer or continuous culture), spontaneous mutations arise in individual cells. If by chance, a spontaneous mutation confers a growth advantage, then descendants of the mutant cell will slowly or quickly (depending on the magnitude of the growth advantage) out-grow the other cells and take over the population. At any time during the metabolic evolution process, individual cells can be isolated from the liquid cultures by streaking on Petri plates containing an equivalent medium plus 2% agar, and individual colonies can be picked and tested in comparison to the starting strain and intermediate isolates.

The preferred starting strain for metabolic evolution is one where unwanted competing pathways have been eliminated by completely deleting relevant genes, so that any unwanted pathway is much less likely to reappear by reversion or suppression. At any given step, a culture can be exposed to a mutagen, for example nitrosoguandine (NTG), ethylmethane sulfonate (EMS), hydrogen peroxide, or ultraviolet radiation, in order to increase the frequency of mutations.

After an economically attractive strain has been obtained by metabolic evolution, its genome can be sequenced by any of the methods well known in the art, for example shotgun cloning and sequencing, the Illumina platform, and the PacBio platform. The resulting DNA sequence can then be compared to that of a starting or ancestor strain, so that every mutation that occurred during metabolic evolution can be identified. Optionally, each mutation can be studied by itself or in combinations by reinstalling the wild type allele(s) into the evolved strain, to study which mutations contribute to the observed improvement in product formation, or by “reverse engineering” in which individual mutations from the genomic sequence, or combinations thereof, are introduced into a naïve or wild type strain.

The above described process has been performed on E. coli strains engineered to produce succinic acid, ethanol, and D-lactic acid, as well as other products (for example, WO Patent application WO2011063055A2). The various software packages that are designed to process the raw genomic sequence data and assemble the raw data into a complete genomic sequence, for example the Lasergene Genomic Suite, typically produce a table of mutations found in the evolved strain when compared to a reference (parent) strain. However, such computer-generated tables can be incomplete or difficult to interpret precisely, especially for DNA sequences that are repeated. For example, E. coli strains typically contain multiple copies of various insertion (IS) elements. For example E. coli Crooks (ATCC 8739), Genbank Accession Number NC_010468, contains many copies of IS4. Since IS4 is about 1400 base pairs, and the Illumina platform sequence reads are only about 50-250 bases in length, and double ended reads are typically from fragments of about 500 base pairs in length, it is impossible for sequencing software to accurately place different variants of IS4, since sequence reads from the middle of an IS4 copy will not overlap with the surrounding DNA. Moreover, divergent alleles in the middle of large duplications that contain many genes, such as the type of duplication disclosed herein, will not be tractable, even with the longer reads possible with the PacBio platform.

Duplications of one or more genes are well known to occur during strain development (Elliott, Cuff et al. 2013). For example, in the development of penicillin producing strains, which was done largely by mutagenesis and screening (as opposed to genetic engineering and metabolic evolution), the penicillin biosynthetic gene cluster was shown to have spontaneously amplified up to five or six tandem copies (Fierro, Barredo et al. 1995). Deliberate amplification of gene cassettes in Bacillus subtilis is a well-known method in which an incoming cassette contains an antibiotic resistance gene, for example a tetracycline resistance gene, that provides limited resistance, and then evolving the resulting transformant for higher resistance by growing the strain on increasing concentrations of the antibiotic (EP 1214420). The resulting strains are then confirmed to have about two to seven copies of the cassette in a tandem duplication. However, such tandem duplications can collapse down to a single copy by homologous recombination if the strain is grown in the absence of antibiotic. On the other hand, growth in the presence of high concentrations of antibiotic is impractical and undesirable.

A similar method was used to amplify copy number of an integrated cassette in E. coli (Tyo, Ajikumar et al. 2009). However, once again an antibiotic resistance gene was used to accomplish the amplification, and the recA gene was deleted to preserve the amplified copy number. It is well known that populations of recA-cells contain a high percent of dead cells, so this solution is not ideal. Amplification of gene copy number has also been accomplished in animal cells, but the copy number was unstable when specific selection pressure was removed (Tyo, Ajikumar et al. 2009).

Amplification of gene cassettes in tandem arrays is also known in Saccharomyces yeast (U.S. Pat. No. 7,527,927, (Lopes, de Wijs et al. 1996)). The cassette to be amplified can contain a sequence homologous to a repeated ribosomal DNA gene and a selectable marker such as an antibiotic G418 resistance gene (U.S. Pat. No. 7,52,927) or an auxotrophy-complementing marker such as the dLEU2 gene (Lopes, de Wijs et al. 1996). However, again, in the absence of a strong selection, the amplified copy number is unstable and is lost (Lopes, de Wijs et al. 1996).

In the penicillin example given above, there was no selectable marker to stabilize and maintain the amplified copy number. Although the stability of the amplified cassette was not discussed, in theory the copy number could be easily lost by homologous recombination. The only means to maintain the amplified copy number would be careful curation and frequent testing of stocks for maintenance of the original productivity. In the other examples, the cassette to be amplified in copy number contains a selectable marker to begin with, but the marker requires special conditions in order to maintain the amplified copy number, either high or expensive concentrations of an antibiotic, or a chemically defined medium. Thus, in all prior art cases known to the inventors, there is no method for stabilizing the copy number of duplicated DNA sequences under desirable culture conditions, namely culture conditions that are inexpensive, practical, and well suited for producing the desired product. There is also no known method for stabilizing duplicated DNA sequences that arose spontaneously, either with or without deliberate selective pressure, where there is no easily usable selectable marker associated with the duplicated DNA sequence. Thus, there is still a need for methods that stabilize and maintain useful copy numbers of tandemly duplicated DNA sequences in microbes engineered for economically attractive commercial production of a chemical, whether the chemical is a commodity such as succinic acid, or a higher value chemical such as particular protein. This invention provides such methods and strains derived from said methods.

Another surprising discovery is that even after extensive metabolic evolution and genome sequencing, a microbial strain can further evolve during routine handling and storage. Such further evolution can result from culturing a strain under fermentation conditions that are different from the fermentation conditions used during the metabolic evolution.

Increased demand for crude oil has resulted in a global effort to generate alternative fuels and chemicals from renewable resources to replace current fuels and petroleum derived chemicals. In 2004, the US Department of Energy developed a list of the top twelve potential chemicals from biomass. One of these chemicals is succinic acid.

Succinic acid can be chemically converted to a wide variety of target compounds well known in the industry, including 1,4-butanediol (BDO), tertahydrofuran (THF), gamma-butyrolactone (GBL) and N-methylpyrrolidone. Succinic acid is also useful in the manufacture of a number of large volume commercial products including animal feed, plasticizers, coalescing agents, congealers, fibers, plastics, and polymers such as PBS (polybutyl succinate). PBS is a biodegradable polymer capable of replacing current polymers that are petroleum derived and not biodegradable.

Succinic acid (C₄H₆O₄), also known as 1,4-butanedioic acid, is a dicarboxylic acid which readily takes the form of succinate anion and has multiple roles in living organisms. Succinate is an intermediate in the tricarboxylic acid (TCA) cycle, an energy producing cycle shared by all aerobic organisms, and is one of the fermentation products produced by many bacteria. Succinate is produced from glucose (C₆H₁₂O₆), a hexose sugar, as a starting material by a series of enzymatically catalyzed reactions, with the following overall stoichiometry: 7 C₆H₁₂O₆+6CO₂>12C₄H₆O₄+6H₂O. The maximum theoretical yield of this reaction when running a redox balanced combination of reductive and oxidative pathways under anaerobic conditions is 1.71 moles of succinic acid per mole of glucose or 1.12 grams of succinic per gram of glucose.

Microbial biocatalysts have been developed for the commercial scale fermentative production of succinic acid using a number of carbon sources. Escherichia coli strain KJ122 was derived from the E. coli Crooks strain by means of introducing mutations in a number of genes involved in the operation of various metabolic pathways (ΔldhA, ΔadhE, ΔackA, ΔfocA-pflB, ΔmgsA, ΔpoxB, ΔtdcDE, ΔcitF, ΔaspC, ΔsfcA) and subjecting the genetically engineered strain to the process of metabolic evolution during various stages of genetic engineering ((Jantama, Haupt et al. 2008); (Jantama, Zhang et al. 2008); Zhu et al. 2014; and U.S. Pat. No. 8,691,539).

During the last several years, E. coli KJ122 has been further improved to use a number of carbon sources other than glucose. KJ122 was subjected to metabolic evolution in the presence of C₅ and C₆ sugars derived from cellulosic hydrolysis to develop a strain that could use both C₅ and C₆ sugars in the succinic acid production (U.S. Pat. No. 8,871,489). KJ122 has also been subjected to genetic engineering to produce a strain that could use sucrose (WO2012/082720) or glycerol (WO2011373671) as the source of carbon for the production of succinic acid. Effort has also been made to improve the efficiency for sugar import as a way of increasing succinic acid production in the KJ122 bacterial strain (WO2015/013334).

Whole genome sequencing had been used in the identification of various mutations that occurred in KJ122 strain during the process of metabolic evolution. Reverse genetic analysis was followed to establish the significance of those mutations identified through whole genome sequencing in the succinic acid production. It has been unexpectedly discovered that a number of KJ122 stocks perform differently in 7 L fermenters and the difference in the performance among these KJ122 stocks was as great as 30% decrease or a 50% increase (depending on which stock was used as the reference stock) in succinate titer. Whole genome sequencing of these different stocks of KJ122 followed by a comparative analysis of genomic sequences of these KJ122 stocks using a Lasergene Genomic Suite software package from DNAStar (Madison, Wis., USA) identified a number of functional DNA sequence duplications in certain genomic regions in some KJ122 stocks, and at least one of these genomic duplications was found to be associated with an increased titer for succinic acid production. However, the desirable genomic duplication was unstable, so a method for stabilizing the useful duplication was needed. The invention provides a way to stabilize the desired genomic duplication in the production strain.

For the foregoing reasons, there is still an unmet need in the art to (1) create large gene duplications that enhance production of a desired product, (2) identify large gene duplications in production strains, (3) determine the precise structure of large gene duplications, and (4) stabilize beneficial large gene duplications with respect to copy number.

Methods and microbial strains are disclosed herein that overcome the existing problems and limitations in the art.

### SUMMARY OF INVENTION

The invention relates to processes for identifying and tracking genomic duplications that can occur during classical strain development or during the metabolic evolution of microbial strains originally constructed for the production of a biochemical through specific genetic manipulations, processes that stabilize the copy number of desirable genomic duplications using appropriate selectable markers, and non-naturally occurring microorganisms with stabilized copy numbers of a functional DNA sequence.

In one embodiment, the invention provides a process involving whole genome DNA sequencing to identify the functional DNA sequence duplications that occur during the metabolic evolution of microbial strains originally engineered for the production of a biochemical through intentional genetic manipulations. In one aspect of this embodiment, the invention involves a comparative genomic analysis involving several isolates and derivatives of a succinic acid producing E. coli strain KJ122 and identification of a tandem duplication of a functional DNA sequence comprising a large multi-gene portion of the genome referred herein as the “B duplication”, which is associated with high titer succinic acid production. In another aspect of this embodiment, the invention has identified and solved the challenges in introducing further genetic modifications in the KJ122 strain with the B duplication.

In another embodiment, the invention provides a process for stabilizing a desirable genomic duplication that occurred during the process of metabolic evolution. In one aspect of this embodiment, the invention provides a process for stabilizing the desirable genomic duplication that occurred during metabolic evolution by inserting a selectable marker between the two adjacent copies of the duplicated genes. The set of selectable markers suitable for this purpose includes, but is not limited to, antibiotics resistance genes, genes coding for one or more proteins involved in the house-keeping functions, essential genes, conditionally essential genes, auxotrophy-complementing genes, and any exogenous gene coding for protein with a selectable phenotype such as the ability to utilize sucrose as a sole source of carbon for growth.

In yet another embodiment, the invention provides a method for constructing and stabilizing a genetically engineered strain with a B duplication for fermentative production of a desirable biochemical. In one aspect of this embodiment, the invention describes the construction of a microbial strain having high-titer for succinic acid production together with a reduced level of acetic acid as a byproduct.

Unless otherwise defined, all terms used herein have the same meaning as commonly understood by one of ordinary skill in the art to which the invention pertains. Although examples of suitable methods and materials for practicing the are described below, those skilled in the art will know that based on the disclosed examples, methods and materials similar or equivalent to those described herein can be used in the practice or testing of the invention. All publications, patent applications, patents, and other references mentioned herein are incorporated by reference in their entirety. In case of conflict, the specification, including nomenclature and definitions, will control. The materials, methods, examples, and drawings included herein are illustrative only and not intended to be limiting.

### DESCRIPTION OF DRAWINGS

FIG. 1 is a graph plotting read frequency (depth of coverage) versus genome base pair position from the Illumina (San Diego, Calif., USA) platform genome sequencing and LaserGene Genomic Suite sequencing software from DNAStar (Madison, Wis., USA) that shows the B duplication by a sudden two-fold increase in read frequency.

FIG. 2 is a diagram depicting a logical hypothetical mechanism for the formation of the B duplication in KJ122 in three steps.

FIG. 3 is a diagram depicting a diagnostic PCR for determining the presence of the B duplication.

FIG. 4 is a diagram depicting a mechanism for stabilizing the B duplication using a selectable marker.

FIG. 5 is a diagram depicting the construction of bacterial strains with glf, glk and the B duplication.

FIG. 6 is a diagram depicting the construction of bacterial strains with rrsG::cscBAK and a stabilized B duplication.

### NOMENCLATURE

To facilitate understanding of the invention, a description of nomenclature is provided below.

In regards to nomenclature, a bacterial gene or coding region is usually named with lower case letters in italics, for example “tpiA” or simply “q3.i” from E. coli are names for the gene that encodes triose phosphate isomerase, while the enzyme or protein encoded by the gene can be named with the same letters, but with the first letter in upper case and without italics, for example “TpiA” or “Tpi”. A yeast gene or coding region is usually named with upper case letters in italics, for example “PDC1”, which encodes a pyruvate de carboxylase enzyme, while the enzyme or protein encoded by the gene can be named with the same letters, but with the first letter in upper case and without italics, for example “Pdc1” or “Pdc1p”, the latter of which is an example of a convention used in yeast for designating an enzyme or protein. The “p” is an abbreviation for protein, encoded by the designated gene. The enzyme or protein can also be referred to by a more descriptive name, for example, triose phosphate isomerase or pyruvate decarboxylase, referring respectively to the two above examples. A gene or coding region that encodes one example of an enzyme that has a particular catalytic activity can have several different names because of historically different origins, functionally redundant genes, genes regulated differently, or because the genes come from different species. For example a gene that encodes glycerol-3-phosphate dehydrogenase can be named GPD1, GDP2, or DAR1, as well as other names.

### Definitions

To facilitate understanding of the invention, a number of terms are defined below, and others are found elsewhere in the specification.

“Microorganism” means any cell or strain of bacterium, yeast, filamentous fungus, or archaea. Microorganisms can be deliberately genetically altered, or allowed to spontaneously change genetically, by using any of one or more well-known methods, for example by mutagenesis, mating, breeding, genetic engineering, evolution, selection and screening. In such a process, a starting strain, which is referred to herein as an “ancestor microorganism” or an “ancestor strain,” is genetically altered to create a new strain that can be propagated by one or more cell divisions from said ancestor strain following acquisition of one or more genetic changes. The strain that is genetically different from the ancestor strain, but derived from said ancestor strain by genetic alteration and subsequent cell division, is referred to herein as a “descendant microorganism” or a “descendant strain”. A descendant microorganism can have one, or more than one, genetic change relative to its ancestor strain. A descendant microorganism can result from any finite number of generations (cell divisions) from its ancestor microorganism. One type of descendant microorganism is a “derivative microorganism”, which means a microorganism created by deliberate addition, removal, or alteration of a DNA sequence in an ancestor microorganism. A descendant microorganisms can be distinguished from its related ancestor microorganism by DNA sequencing or by any other measurable phenotype, such as an improved fermentation parameter.

“Cassette,” “expression cassette,” or “gene cassette” means a deoxyribonucleic acid (DNA) sequence that is capable of causing or increasing the production of, or alternatively eliminating or lowering of the production of, one or more desired proteins, enzymes, or metabolites when installed in a host organism. A cassette for producing a protein or enzyme typically comprises at least one promoter, at least one protein coding sequence, and optionally at least one terminator sequence. If a gene to be expressed is heterologous or exogenous, the promoter and terminator are usually derived from two different genes or from a heterologous gene, in order to prevent double recombination with the native gene from which the promoter or terminator was derived. A cassette can optionally and preferably contain one or two flanking sequence(s) on either or both ends that is/are homologous to a DNA sequence in an ancestor (or “host” or “parent”) organism, such that the cassette can undergo homologous recombination with the genome of the host organism, either with a chromosome or a plasmid, resulting in integration of the cassette into said chromosome or plasmid at the site that is homologous to said flanking sequences. If only one end of the cassette contains a flanking homology, then the cassette in a circular format can integrate by single recombination at the flanking sequence. If both ends of a cassette contain flanking homologies, then the cassette supplied in a linear or circular format can integrate by double recombination with the surrounding flanks, or the circular form can integrate in its entirety by a single crossover event. A cassette can be constructed by genetic engineering, where, for example, a coding sequence is expressed from a non-native promoter, or the cassette can use the naturally associated promoter. A cassette can be built into a plasmid, which can be circular, or it can be a linear DNA created by restriction enzyme cutting, polymerase chain reaction (PCR), primer extension PCR, or by in vivo or in vitro homologous recombination. A cassette can be designed to include a selectable marker. A cassette can be constructed in one or more steps using methods ell known ion the art, for example joining of restriction enzyme-generated fragments by ligation, the “Gibson method” using a NEBuilder kit (New England BioLabs, Ipswitch, Mass., USA), and in vivo assembly.

“Selectable marker” means any gene, cassette, or other form of DNA sequence that does not functionally exist in a parent or ancestor microbial strain, but which can be installed into said ancestor microbial strain, can function after being installed in said ancestor microbial strain to produce a type of descendant strain, and is required for growth of said descendant strain under at least one set of growth conditions. As such, a selectable marker, either by itself, or when included in a cassette together with one or more other useful DNA sequences, can be used to select or screen for successful installation of said selectable marker with or without an additional attached DNA sequence, upon transformation, transduction, transfection, breeding, or mating, into a strain that did not previously contain said selectable marker. In some cases, a selectable marker can be mutated in (preferably deleted from) a strain, whereupon an unmutated version can then be used as a selectable marker in the resulting mutated or deleted strain. In most cases, an ancestor strain without the selectable marker is able to grow under a particular set of conditions (for example a rich medium, a nutrient supplemented medium, or a medium lacking an antibiotic), but after the selectable marker is installed in to the parent strain, the progeny or descendant strain is able to grow under a set of conditions where the parent strain cannot grow (for example a minimal medium, a medium lacking a nutrient, or a medium containing an antibiotic). Useful selectable marker genes include, but are not limited to, functional antibiotic resistance genes or cassettes, genes or cassettes that confer growth on a particular carbon source such as sucrose or xylose, and biosynthetic pathway genes that can be deleted under certain growth conditions (for example, tpiA in E. coli, which is essential in a minimal glucose or minimal sucrose medium, but is not essential in a rich medium such as Luria broth). For a biosynthetic pathway gene (for example, pyrF or URA3) to be used as a selectable marker, the parent strain must, of course, contain a mutation in the corresponding gene, preferably a null mutation (a mutation that causes effective loss of function). For an antibiotic resistance gene, the resistance gene usually requires a promoter that functions well enough in the host strain to enable selection. Although a selectable marker that is desired to be expressed can be installed in a host strain in the form of a cassette, a DNA sequence, for example a coding sequence from start codon to stop codon, can be integrated into a host chromosome or plasmid without a promoter or terminator such that the incoming coding sequence precisely or approximately replaces the coding sequence of a gene native to the host strain, such that after integration, the incoming coding region is expressed from the remaining promoter that had been associated with the host coding sequence that was replaced by the incoming coding sequence.

“Transformant” means a cell or strain that results from installation of a desired DNA sequence, either linear or circular, and either autonomously replicating or not, into a host or parent or ancestor strain that did not previously contain said DNA sequence. “Transformation” means any process for obtaining a transformant.

“Titer” means the concentration of a compound in a fermentation broth, usually expressed as grams per liter (g/l) or as percent weight per volume (% w/v). Titer is determined by any suitable analytical method, such as quantitative analytical chromatography, for example high pressure liquid chromatography (HPLC) or gas chromatography (GC), with a standard curve made from external standards, and optionally using an internal standard.

“Heterologous” means a gene or protein that is not naturally or natively found in an organism, but which can be introduced into an organism by genetic engineering, such as by transformation, mating, transfection, or transduction. A heterologous gene can be integrated (i.e., inserted or installed) into a chromosome, or contained on a plasmid. “Exogenous” means a gene or protein that has been introduced into, or altered, in an organism for the purpose of increasing, decreasing, or eliminating an activity relative to that activity of a parent or host strain, by genetic engineering, such as by transformation, mating, transduction, or mutagenesis. An exogenous gene or protein can be heterologous, or it can be a gene or protein that is native to the host organism, but which has been altered by one or more methods, for example, mutation, deletion, change of promoter, change of terminator, duplication, or insertion of one or more additional copies in the chromosome or in a plasmid. Thus, for example, if a second copy of a DNA sequence is inserted at a site in the chromosome that is distinct from the native site, the second copy would be exogenous.

“Plasmid” means a circular or linear DNA molecule that is substantially smaller than a chromosome, is separate from the chromosome or chromosomes of a microorganism, and replicates separately from the chromosome or chromosomes. A plasmid can be present in about one copy per cell or in more than one copy per cell. Maintenance of a plasmid within a microbial cell usually requires growth in a medium that selects for presence of the plasmid, for example using an antibiotic resistance gene, or complementation of a chromosomal auxotrophy. However, some plasmids require no selective pressure for stable maintenance, for example the 2 micron circle plasmid in many Saccharomyces strains.

“Chromosome” or “chromosomal DNA” means a linear or circular DNA molecule that is substantially larger than a plasmid and usually does not require any antibiotic or nutritional selection for maintenance. In this invention, a yeast artificial chromosome (YAC) can be used as a vector for installing heterologous and/or exogenous genes, but it would usually require selective pressure for maintenance.

“Overexpression” means causing the enzyme or protein encoded by a gene or coding region to be produced in a parent or host microorganism at a level that is higher than the level found in the wild type version of the parent or host microorganism under the same or similar growth conditions. This can be accomplished by, for example, one or more of the following methods: installing a stronger promoter, installing a stronger ribosome binding site, installing a terminator or a stronger terminator, improving the choice of codons at one or more sites in the coding region, improving the mRNA stability, or increasing the copy number of the gene either by introducing multiple copies in the chromosome or placing the cassette on a multicopy plasmid. An enzyme or protein produced from a gene that is overexpressed is said to be “overproduced.” A gene that is being overexpressed or a protein that is being overproduced can be one that is native to a host microorganism, or it can be one that has been transplanted by genetic engineering methods from a different organism into a host microorganism, in which case the enzyme or protein and the gene or coding region that encodes the enzyme or protein is called “foreign” or “heterologous.” Foreign or heterologous genes and proteins are by definition overexpressed and overproduced, since they are not present in the unengineered host organism.

“Homolog” means a first gene or DNA sequence that has at least 50% sequence identity when compared to a second DNA sequence, or at least 25% amino acid sequence identity when said first DNA sequence is translated into a first protein sequence and said first protein sequence is compared to a second protein sequence derived by translating said second DNA sequence, as determined by the Basic Local Alignment Search Tool (BLAST) computer program for sequence comparison (Altschul, Gish et al. 1990, Altschul, Madden et al. 1997), and allowing for deletions and insertions. If a first DNA or protein sequence is found to be a homolog of a second DNA or protein sequence, then the two sequences are said to be “homologs” or “homologous.” When a cassette is intended to integrate into a genome at a specific site, it is preferable that the flanking homologous DNA sequence(s) have 100% identity or almost 100% identity to the DNA sequence being targeted.

“Analog” means a gene, DNA sequence, or protein that performs a similar biological function to that of another gene, DNA sequence, or protein, but where there is less than 25% sequence identity (when comparing protein sequences or comparing the protein sequence derived from gene sequences) with said another gene, DNA sequence, or protein, as determined by the BLAST computer program for sequence comparison (Altschul et al. 1990; Altschul et al. 1997), and allowing for deletions and insertions. An example of an analog of a Saccharomyces cerevisiae Gpd1 protein would be a S. cerevisiae Gut2 protein, since both proteins are enzymes that catalyze the same reaction, but there is no significant sequence homology between the two enzymes or their respective genes. A person having ordinary skill in the art will know that many enzymes and proteins that have a particular biological function (in the immediately above example, glycerol-3-phosphate dehydrogenase), can be found in many different organisms, either as homologs or analogs, and since members of such families of enzymes or proteins share the same function, although they may be slightly or substantially different in structure. Different members of the same family can in many cases be used to perform the same biological function using current methods of genetic engineering. Thus, for example, a gene that encodes triosephosphate isomerase could be obtained from any of many different organisms.

“Mutation” means any change from a native or parent or ancestor DNA sequence, for example, an inversion, a duplication, an insertion of one or more base pairs, a deletion of one or more base pairs, a point mutation leading to a base change that creates a premature stop codon, or a missense mutation that changes an amino acid encoded at that position. A mutation can even mean a single or multiple base pair change in a DNA sequence that does not lead to a change in a predicted amino acid sequence encoded by said DNA sequence. “Null mutation” means a mutation that effectively eliminates the function of a gene. A complete deletion of a coding region would be a null mutation, but single base changes can also result in a null mutation. “Mutant,” “mutant strain,” “mutated strain,” or a strain “that has been mutated” means a strain that comprises one or more mutations when compared to a native, wild type, parent or ancestor strain.

“A mutation that eliminates or reduces the function of” means any mutation that lowers any assayable parameter or output, of a gene, protein, or enzyme, such as mRNA level, protein concentration, metabolite production, or specific enzyme activity of a strain, when said assayable parameter or output is measured and compared to that of the unmutated parent strain grown under similar conditions. Such a mutation is preferably a deletion mutation, but it can be any type of mutation that accomplishes a desired elimination or reduction of function.

“Strong constitutive promoter” means a DNA sequence that typically lies upstream (to the 5′ side of a gene when depicted in the conventional 5′ to 3′ orientation), of a DNA sequence or a gene that is transcribed by an RNA polymerase, and that causes said DNA sequence or gene to be expressed by transcription by an RNA polymerase at a level that is easily detected directly or indirectly by any appropriate assay procedure. Examples of appropriate assay procedures include quantitative reverse transcriptase plus PCR, enzyme assay of an encoded enzyme, Coomassie Blue-stained protein gel, or measurable production of a metabolite that is produced indirectly as a result of said transcription, and such measurable transcription occurring regardless of the presence or absence of a protein that specifically regulates the level of transcription, a metabolite, or an inducer chemical. By using well-known methods, a strong constitutive promoter can be used to replace a native promoter (a promoter that is otherwise naturally existing upstream from a DNA sequence or gene), resulting in an expression cassette that can be placed either in a plasmid or chromosome and that provides a level of expression of a desired DNA sequence or gene at a level that is higher than the level from the native promoter. A strong constitutive promoter can be specific for a species or genus, but often a strong constitutive promoter from a yeast can function well in a distantly related yeast. For example, the TEF1 (translation elongation factor 1) promoter from Ashbya gossypii functions well in many other yeast genera, including Saccharomyces cerevisiae.

“Microaerobic” or “microaerobic fermentation conditions” means that the deliberate supply of air to a fermentation tank is less than 0.1 volume of air per volume of liquid broth per minute (vvm). “Anaerobic” or “anaerobic fermentation conditions” means that no air is deliberately supplied to a fermentation tank. “Aerobic” or “aerobic fermentation conditions” means that 0.1 or more volume of air per volume of liquid broth per minute (vvm) is deliberately supplied to a fermentation tank. Classically, “fermentation” referred to anaerobic or microaerobic cultures of microorgansims. However, for simplicity in this specification, the term “fermentation” or “fermentation conditions” means any type of growth or culture of a microorganism, including anaerobic, microaerobic, or aerobic, and including growth in a liquid medium or on a solid medium, for example on an agar Petri plate. “Fermentor” means any container in which fermentation is or can be performed. In shake flask cultures (also known as shaking flask cultures), where aeration conditions are not precisely controlled, the fermentation conditions can be anaerobic, microaerobic, or aerobic, and the conditions can change during the course of a culture. For example, at the beginning of a shake flask culture with a low level of inoculum (for example, a starting OD600 of 0.5 or less), and vigorous shaking (for example 200 rpm or greater), and a loose fitting or porous cap, the fermentation conditions are likely to be aerobic. However, as the culture grows to a higher density (for example, an OD600 of 10 or greater), the consumption of oxygen by the microorganism can be large relative to the rate at which oxygen enters the flask, resulting in anaerobic or microaerobic conditions. The fermentation conditions in a shake flask can be forced to be anaerobic or microaerobic by using an air trap (for example, a bubbler that allows escape of carbon dioxide), or a cap or closure that is impermeable to air. It should be understood that unless stringent conditions are used (for example, gassing with nitrogen, carbon dioxide, or argon), strictly anaerobic conditions may not be attained, so that there is a continuum between anaerobic and microaerobic fermentation conditions. Thus, the terms anaerobic and microaerobic are usually used together herein.

“Duplication” means a functional DNA sequence that is present in n copies per haploid genome in a descendant microorganism, after being present in n−1 copies per haploid genome in the related ancestor microorganism, where n is an integer greater than or equal to two. The term “duplication” refers to all n or more copies of said functional DNA. “Duplicated DNA,” “DNA that is duplicated,” or “the duplicated DNA sequence” means any one single copy of said functional DNA. In a duplication, the ends of the duplicated DNA copies might differ among the various copies. Thus, for the example of the B duplication disclosed herein, one copy of the functional DNA sequence ends with an IS4 insertion element in the middle of the menC coding sequence, while the second copy ends with an intact copy of the menC gene. The two or more copies of duplicated DNA can contain minor differences in their sequences. A duplication in which the two or more copies are adjacent to each other or nearly adjacent to each other is referred to as a “tandem duplication.” For clarification, the terms “duplication” and “duplicated DNA” do not refer to extra copies of said functional DNA that are normally created during the replication of a microorganism's genome as a normal precursor to cell division or cell budding.

“Similar culture conditions” or “similar fermentation conditions” means conditions designed to compare the performance of two different microorganisms, in which the experimenter attempts to set up and control all conditions in the two culture to be as identical as possible. The term “similar” is used herein because it is well known that condition in two separate microorganism cultures cannot be practically made to be absolutely identical.

“Fermentation parameter” means one of several measurable aspects of a fermentation or culture, for example, time to completion, temperature, pH, titer of product in grams/liter (g/l), yield in grams product/grams input nutrient, or specific productivity (grams product/grams cell mass per hour. A fermentation parameter is said to be “improved” for a descendant microorganism when compared to the related ancestor microorganism if one or more fermentation parameters is economically more favorable for the descendant microorganism, when both microorganisms are cultures under similar culture conditions. Examples of improved fermentation parameters are increased titer, yield, or specific productivity, or a decreased time to completion.

“Chemically defined medium,” “minimal medium,” or “mineral medium” means any growth medium that is comprised of purified or partially purified chemicals such as mineral salts (for example, sodium, potassium, ammonium, magnesium, calcium, phosphate, sulfate, and chloride) which provide a necessary element such as nitrogen, sulfur, magnesium, phosphorus (and sometimes calcium and chloride), vitamins (when necessary or stimulatory for the microbe to grow), one or more purified carbon sources, such as a sugar, glycerol, ethanol, methane, trace metals, as necessary or stimulatory for the microbe to grow (such as iron, manganese, copper, zinc, molybdenum, nickel, boron, and cobalt), and, optionally, an osmotic protectant such as glycine betaine, also known as betaine. Except for the optional osmoprotectant and vitamin(s), such media do not contain significant amounts of any nutrient or mix of more than one nutrient that is not essential for the growth of the microbe being fermented. If a microorganism is an auxotroph, for example, an amino acid or nucleotide auxotroph, then a minimal medium that can support growth of said auxotroph will necessarily contain the required nutrient, but for a minimal medium, the added nutrient will be in a substantially pure form. A minimal medium does not contain any significant amount of a rich or complex nutrient mixture, such as yeast extract, peptone, protein hydrolysate, molasses, broth, plant extract, animal extract, microbe extract, whey, and Jerusalem artichoke powder. For producing a commodity chemical by fermentation where purification of the desired chemical by simple distillation is a not an economically attractive option, a minimal medium is preferred over a rich medium.

“Fermentation production medium” means the medium used in the last tank, vessel, or fermentor, in a series comprising one or more tanks, vessels, or fermentors, in a process wherein a microbe is grown to produce a desired product (for example succinic acid). For production of a commodity chemical by fermentation such as succinic acid, where extensive purification is necessary or desired, a fermentation production medium that is a minimal medium is preferred over a rich medium because a minimal medium is often less expensive, and the fermentation broth at the end of fermentation usually contains lower concentrations of unwanted contaminating chemicals that need to be purified away from the desired chemical. Although it is generally preferred to minimize the concentration of rich nutrients in such a fermentation, in some cases it is advantageous for the overall process to grow an inoculum culture in a medium that is different from the fermentation production medium, for example to grow a relatively small volume (usually 10% or less of the fermentation production medium volume) of inoculum culture grown in a medium that contains one or more rich ingredients. If the inoculum culture is small relative to the production culture, the rich components of the inoculum culture can be diluted into the fermentation production medium to the point where they do not substantially interfere with purification of the desired product. A fermentation production medium must contain a carbon source, which is typically a sugar, glycerol, fat, fatty acid, carbon dioxide, methane, alcohol, or organic acid. In some geographic locations, for example in the Midwestern United States, D-glucose (dextrose) is relatively inexpensive and therefore is useful as a carbon source. Most prior art publications on lactic acid production by a yeast use dextrose as the carbon source. However, in some geographic locations, such as Brazil and much of Southeast Asia, sucrose is less expensive than dextrose, so sucrose is a preferred carbon source in those regions.

“Unequal crossover” means a mechanism by which a DNA sequence becomes duplicated or by which duplicated DNA sequence becomes unduplicated. Unequal crossover usually occurs during DNA replication, when there are two copies of a duplicated DNA sequence are in close proximity to each other (Reams and Roth, 2015).

“Functional DNA sequence” is any DNA sequence that produces, either by itself, or when attached in series to another DNA sequence, a measurable phenotype or output when present in an organism's genome. An example of a functional DNA sequence is the section of the chromosome of strain KJ122-RY comprising the 111 genes that are present in one copy in strain KJ122-RY and are present in two copies (present in the B duplication) in strain KJ122-F475 (see Tables 1 and 2). The measurable phenotype or output is, when a second copy is present, the succinic acid titer in typical comparative fermentations increases from about 57 g/L to about 89 g/L. Another example of a functional DNA sequence is the penicillin biosynthetic gene cluster that is amplified in penicillin production strains (Fierro, Barredo et al. 1995). In this case, the phenotype or output is increased penicillin production.

### DESCRIPTION OF INVENTION

In one embodiment, the invention provides a method for increasing the titer of a desired fermentation product, comprising metabolically evolving a strain under a first set of conditions, for example anaerobic or microaerobic conditions, and then growing the resulting evolved strain under a set of second conditions, for example aerobically, to allow further evolution, and screening among strains isolated from the second conditions for strains that are improved for production of the desired fermentation product.

In another embodiment, the invention provides for methods for identifying and tracking genomic duplications that lead to improved production of a desired fermentation product.

In another embodiment, the invention provides for methods for stabilizing beneficial genomic duplications.

In another embodiment, the invention provides for non-naturally occurring microorganisms with stabilized copy numbers of a functional DNA sequence.

A two-step process can be followed in the generation of a microbial strain for the production of a valuable biochemical through biological fermentation. In the first step, rationally-designed genetic modifications are introduced into the microbial cell. In the second step, the genetically modified microbial cell is subjected to metabolic evolution to obtain a microbial catalyst with a desirable phenotype. For example, in the case of developing a bacterial strain for producing succinic acid, a number of genetic modifications were introduced to direct the path of carbon within the microbial cell towards succinic acid production. The intentional genetic modifications are designed on the basis of our knowledge of metabolic pathways in the microbial cells. The desired genetic modifications are carried out in stages. In between the stages in genetic modifications and at the end of all of the relevant genetic modifications, the microbial strains can be subjected to metabolic evolution to allow the microbial cell population to attain the desired phenotype, namely increases in growth, titer and rate of production of the desired biochemical. In other words, when growth is coupled to production of a particular chemical, selection for faster growth (metabolic evolution) leads to faster production of that chemical.

The process of metabolic evolution is expected to produce particular mutations that favor the desirable phenotype, namely more favorable production parameters for the desired product. Thus at the end of metabolic evolution, the microbial strain will have acquired certain specific genetic modifications. In prior art examples, these genetic modifications have been shown to include simple nucleotide changes, insertions, and deletions of significant portions of the genome. Dramatic declines in the cost of the genome sequencing have made it possible to sequence the whole genome of the metabolically evolved strains to confirm that the originally introduced specific mutations are still retained and to identify the mutations that have been acquired during the process of metabolic evolution.

Once the mutations that occurred in a microbial strain during the metabolic evolution are identified, it is possible to confirm the functional significance of identified mutations through reverse genetic analysis. Reverse genetic analysis is easy to carry out when the mutation is in a single gene.

When the mutation is a major genomic duplication that has occurred during the metabolic evolution or during the subsequent culturing under a second set of conditions, the reverse genetic analysis is difficult or impossible. However, if the major genomic duplication is found to be closely associated with a desirable phenotype through comparative genomic and phenotypic analysis among closely related microbial strains, it becomes desirable to maintain this gene duplication from being lost during the subsequent culturing and large scale use of the strain. The first step in establishing a method to stably maintain a DNA duplication is to understand the precise structure of the duplication and the mechanism(s) that led to the duplication. Once the inventors understood the structure and molecular mechanism that led to the instant major genome duplication and the resulting structure, it became possible to further genetically engineer strains to stably maintain the duplication. It was also useful to develop a polymerase chain reaction (PCR) based diagnostic method to detect the presence or absence of the gene duplication in a microbial strain in order to easily demonstrate that stabilization has been achieved.

One novel way to stably maintain an otherwise unmarked gene duplication (a duplication lacking a convenient or practical selectable marker) is to insert a selectable marker (a DNA sequence such as a gene, a group of genes, or an operon, that can be selected for under at least one culture condition) between an adjacent pair of the duplicated DNA sequences, without disturbing the expression of any of the genes within the duplicated sequences. One type of selectable marker that can be easily introduced at the region of genomic duplication is a gene that encodes antibiotic resistance, also known as an antibiotic resistance gene or an antibiotic resistance marker. A number of readily useful antibiotics resistance genes are well known in the art. In E. coli, there are well known genes that confer resistance to, for example, but not limited to, a penicillin (for example ampicillin), tetracycline, kanamycin, chloramphenicol, streptomycin, spectinomycin, or erythromycin. However, as mentioned above, it is generally undesirable to use an antibiotic resistance gene for large scale fermentations. A preferred selectable marker suitable for the present purpose is an endogenous (native) or exogenous gene that codes for a protein that is essential or conditionally essential. In this case, the wild type gene is mutated or deleted from its native locus, either before or after the gene is inserted at the appropriate sight in the duplication, for example at the junction between the two copies of the duplicated genes. For example, the native tpiA gene that encodes triose phosphate isomerase can be deleted or substantially inactivated my mutation through genetic engineering or classical genetics methods. The microbial cell with an inactivated endogenous tpiA gene cannot grow in a minimal medium containing glucose or another sugar as a source of carbon; however, a tpiA mutant can be propagated in a rich medium such as LB (Luria Broth). Once an exogenous tpiA gene cassette (a cassette designed to integrate at a non-native locus) is inserted into a gene duplication in a strain that lacks a functional tpiA gene, the descendant strain will regain the ability to grow in a minimal medium comprising glucose or other sugar as a source of carbon and as a result, the gene duplication will be stably maintained. Yet another approach for stably maintaining the gene duplication involves the use of an exogenous gene coding for a protein or an operon or other set of genes that encode a set of proteins that confer a selectable phenotype. For example, if the microbial strain that has acquired the gene duplication lacks the ability to utilize sucrose as a carbon source due to absence of a gene or genes coding for one or more proteins involved in metabolism of sucrose, a gene cassette coding for one or more proteins required for sucrose utilization can be inserted into the gene duplication and the duplication can then be stably maintained by growing the resulting strain in a medium containing sucrose as the sole carbon source.

The decision to use a particular selectable marker to stably maintain the gene duplication depends on the totality of the circumstances. For example, when glucose is the desired carbon source, use of sucrose utilization genes as the selectable marker will not be appropriate. Although an antibiotic resistance gene can function well to stabilize a gene duplication, in general it is preferred to use an essential or a conditionally essential gene such as tpiA as the selectable marker so as to avoid the need to use a costly antibiotic, and to avoid large scale production of a potentially transmissible antibiotic resistance gene.

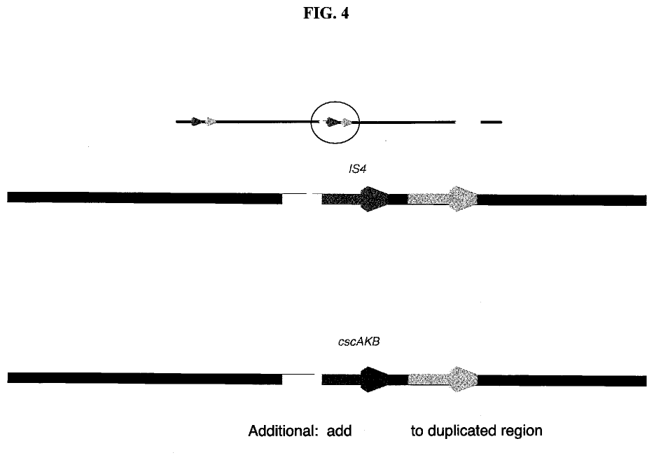

Metabolic evolution is typically performed under a particular set of conditions, such as anaerobic or microaerobic fermentations in a minimal medium (for example see Jantama et al, 2008a; Jantama et al, 2008b; (Zhu, Tan et al. 2014); U.S. Pat. Nos. 8,691,539; 8,871,489; WO Patent application WO2011063055A2). However, in the instant invention, it was discovered that subjecting an evolved strain to a second set of conditions, for example aerobic culturing, can unexpectedly lead to further beneficial evolution that would not be expected to occur without the growth under the second set of conditions. In the example given below, a large duplication of 111 genes (the “B duplication”) occurred during a second set of fermentation conditions in a culture of an ancestor strain KJ122-RY, and the duplication was unlikely to have occurred if the strain had not been grown under the second set of conditions. The only logical series of events that could lead to the B duplication are as follows. First, an transposable IS4 element inserted itself in the middle of the menC gene. The menC gene is the fifth gene in the menFDHBCE operon, which encodes enzymes that are necessary for biosynthesis of menaquinone. Menaquinone is an electron carrier used by E. coli and other microbes during anaerobic growth. A mutation in menC leads to poor anaerobic growth on a minimal glucose medium (Guest 1977). Since the metabolic evolution of KJ122 was conducted under microaerobic conditions in a minimal glucose medium, it is unlikely that the IS4 insertion into menC occurred during that evolution, since a menC null mutant would be at a growth disadvantage. In the second step leading to the B duplication, the 111 gene region between the copy of IS4 annotated as EcolC 1276 in the Genbank version of the E. coli Crooks genome (Accession Number NC_010468), and the copy of IS4 in the menC gene, was precisely duplicated, presumably by unequal crossover between the two aforementioned copies of IS4. This resulting intermediate strain would still be lacking a functional menC gene, so again, it is unlikely that this event would have occurred during the microaerobic evolution. Consistent with this is the fact that the original isolate of KJ122, obtained from a research university laboratory and herein named KJ122-RY, did not have the B duplication. In the third step, the IS4 element in menC at the end of the second copy of the B duplication excised precisely to recreate a functional menC gene, allowing the resulting strain, KJ122-F475, to grow well under microaerobic conditions (see FIG. 4 for a diagrammatic version of these steps). It is the standard practice of the inventors to grow strains such as KJ122-RY and KJ122-F475 aerobicallly on Petri plates and in shake flasks in a minimal glucose medium to make −80° C. freezer stocks, and to make inocula for 7 liter fermentations. As such we believe that the first two steps leading to the B duplication must have occurred during those aerobic growth periods. Since the B duplication has arisen independently on at least three occasions, the inventors deduced that there must have been selective pressure for the duplication to occur, for example a selective advantage for having two copies of one or more of the genes contained in the duplicated DNA sequence under the second set of fermentation conditions. The third step probably occurred during a shake flask cultivation at a time when the dissolved oxygen concentration was relatively low, and microaerobic conditions prevailed, leading to pressure to regenerate a functional copy of the menC gene. While the series of events that led to the formation of the B duplication resulted from culturing the microorganism under microaerobic conditions followed by culturing the microorganism under aerobic conditions, the final step leading to the novel strain KJ-122-F475 (see below), namely precise excision of the IS4 element from the menC gene, likely happened when the strain was subjected to microaerobic conditions again. Thus, beneficial genetic events such as the formation and establishment of the B duplication in its final form, can result from culturing the microorganism first under microaerobic conditions and then subsequently under aerobic conditions, or from culturing the microorganism first under aerobic conditions and then subsequently under anaerobic or microaerobic conditions.

Once a microbial strain with the stable gene duplication associated with a desirable phenotype is generated, that strain can be used as a starting point to construct improved microbial strains for the commercial production of a value added chemical. In another approach, it is also possible to introduce the gene duplication associated with a desirable phenotype into a microbial strain already genetically engineered to produce a value added biochemical, for example by mating, or to introduce additional desirable traits into strains such as KJ122-F475 that already contain the duplication.

### EXAMPLES

### Example 1

### Genome Structure of Various KJ122 Stocks and Phage Resistant Derivatives

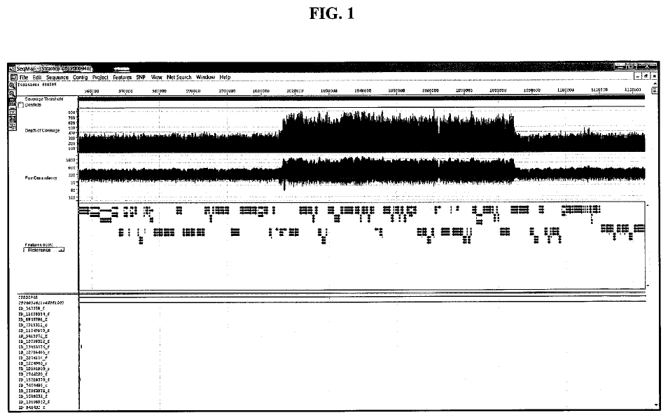

Table 1 provides a summary of genome structures and the succinic acid titers achieved by various different stocks descended from the original E. coli KJ122 strain, including several phage resistant derivatives. All seven strains listed in the Table 1 were subjected to whole genome sequence using technology from Illumina, Inc. (San Diego, Calif., USA) and the genomic data were analyzed using a Lasergene Genomic Suite software package (DNAStar, Madison, Wis.). From the DNA sequence data analysis, it became evident that a particular stock named “KJ122-F475” (sometimes referred to as “KJ122-F”) had unexpectedly acquired two multi-gene duplications when compared to its parent strain, E. coli Crooks (FIG. 1). These two multi-gene duplications are referred to herein as the “A duplication” and the “B duplication”. We also refer to strains comprising the B duplication herein as being “B+”.

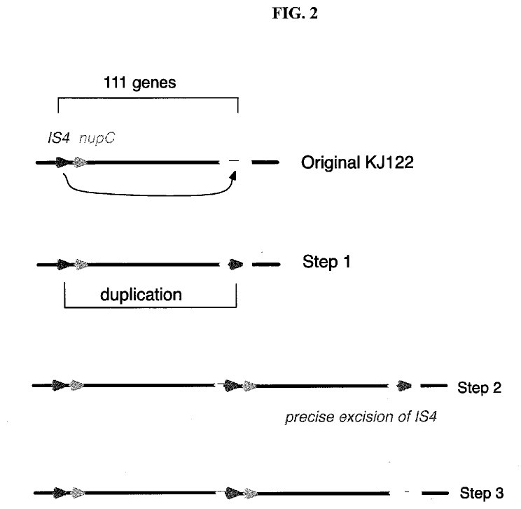

The A duplication included 66 genes and the B duplication included 111 genes. An insertion element IS4 was found at the junction of the repeated sequences in the B duplication (FIG. 2). IS4 is capable of making a copy of itself and inserting that copy at a random location in the chromosome. The pattern of succinate titer and presence or absence of the A duplication and/or B duplication shown in Table 1 strongly suggested that the B duplication was solely responsible for the higher succinate titers produced by some of the strains, for example by KJ122-F475. Furthermore, when strain MH141 (see WO2015/013334), which relies on facilitated diffusion for glucose import and produces significantly less acetate byproduct, was subjected to metabolic evolution to give new strain FES33, the only change revealed by genomic DNA sequencing was the acquisition of precisely the same B duplication. Since FES33 consistently gave higher succinate titers in fed batch fermentations, this observation gave further support to the claim that the B duplication contributes to higher succinate titers. Finally, strain MYR585-4E, a phage resistant derivative of KJ122-RY, independently acquired the B duplication and the increase in succinate titer, giving yet further support for the hypothesis that the B duplication is responsible for the increase in succinate titer.

Table 2 lists various strains relating to the instant invention. Since it was desirable to combine the B duplication with an ability to grow on sucrose as the sole carbon source, an attempt was made to combine the rrsG::cscBAK feature of strain SD14 (KJ122-RY rrsG::cscBAK; see WO2012/082720). The first attempt to combine the two features was made by growing Plvir transducing phage on SD14 and transducing to KJ122-F475 with selection on minimal sucrose plates. Many sucrose+trsnsductants were obtained, but all had lost the B duplication (shown by diagnostic PCR using primers BY296 and BY297), demonstrating that the B duplication was unstable and could be lost, presumably by simple homologous recombination (looping out) of, or unequal crossover between, the two copies of the duplication. A second attempt, which was successful, used well known recombinant DNA transformation methods to transfer the rrsG::cscBAK allele from SD14 to KJ122-F475, namely installation of the phage lambda red recombination system on a plasmid, pKD46 (see Table 3) followed by transformation with linear DNA containing flanking homology to the integration target site (Jantama et al., 2008a; Jantama et al. 2008b). This successful demonstrated that the rrsG::cscBAK allele and the B duplication were not fundamentally incompatible.

Given the potential instability and loss of the B duplication, it became desirable to invent a method for stabilizing the B duplication against loss. As disclosed herein, the desired stabilization can be achieved by inserting a selectable marker, such as an essential gene cassette, or a conditionally essential gene cassette, at the junction between the two copies of the B duplication, such that collapse of the B duplication back to one copy would lead to loss of the inserted selectable marker. Concrete examples of conditionally selectable markers are (1) the cscBAK operon, which, in the absence of sucrose utilization genes in the strain background, is essential for growth on a minimal sucrose medium (see WO2012/082720), and (2) a gene that encodes triose phosphate isomerase, such as the tpiA gene of E. coli Crooks, which is essential for growth on a minimal glucose medium, but which is not essential for growth on a rich medium such as LB (Luria Broth). A person skilled in the art will know that a wide variety of selectable markers and/or gene cassettes and/or operons can be used in a fashion similar to that described herein for stabilization of a tandem gene duplication or a tandem multigene duplication. The only requirement is that the selectable marker or gene cassette be essential for growth under at least one growth condition (i.e., it is essential or conditionally essential). Other examples are housekeeping genes, antibiotic resistance genes, genes that complement an auxotrophy, and genes that confer ability to grow on a nutrient source that the host strain cannot grow on, for example, sucrose, xylose, urea, acetamide, or sulfate. A person skilled in the art will also know that the selectable marker need not be native to the parent organism. For example, a gene or DNA sequence that encodes triose phosphate isomerase from a heterologous organism that can function in the parent or host organism can be used as the selectable marker.

### Example 2

### PCR Diagnosis of the B Duplication in Succinic Acid Production Strains

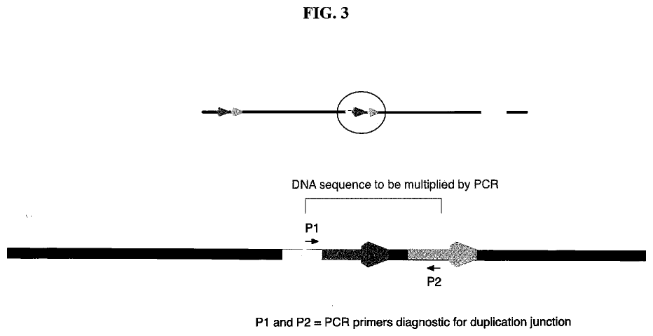

Two primers (BY296 and BY297) were designed for the PCR (polymerase chain reaction) diagnosis of the B duplication. These two primers hybridize just upstream and downstream of the junction site of the B duplication, respectively, as illustrated in FIG. 3. BY296 primes from the 3′ end of the E. coli C 1386 gene (35 bp upstream of from the stop codon), a gene near the 3′ end of the B duplication. BY297 is located at the 3′ end of the E. coli C 1277 gene, the second gene of the B duplication.

The PCR was performed in 50 μl of total working volume with 1 microliter of DNA template (from a single colony or liquid cell culture), two primers (BY296 and BY297), Quick-Load® Taq 2× Master Mix (New England Biolabs, Ipswitch, Mass., USA), and PCR-grade water, as recommend by the NEB manufacturer. The PCR program includes an initial denaturing step of three minutes at 94° C. followed by 35 cycles of 94° C. for 30 seconds, 55° C. for 30 seconds and 68° C. for two minutes, and then a final extension at 68° C. for ten minutes. PCR products were analyzed by agarose gel electrophoresis. A 1788 bp fragment is produced from the B duplication positive strains, and no fragment is produced by the negative control strain, or a control PCR with no template added. The 1788 bp PCR product showed that the B duplication is a tandem duplication, where the two copies are adjacent. The PCR product and genomic DNA sequencing showed that an IS4 insertion element exists at the junction between the two copies of the B duplication, which in turn suggests the mechanism by which the duplication could arise (see FIG. 2). A control PCR reaction, using appropriate primers that produce a similar, but measurably different sized, fragment from a sequence not included in the duplication, can be used to show that the PCR method and conditions are working properly.

### Example 3

### Details about Various Bacterial Strains

Details about various bacterial strains constructed in the invention are provided in Table 2. A list of plasmids used in the invention is provided in Table 3. Sequence information about primers and genes is provided in Tables 4 and 5.

### Example 4

### Construction of Strains Comprising a Stabilized B Duplication

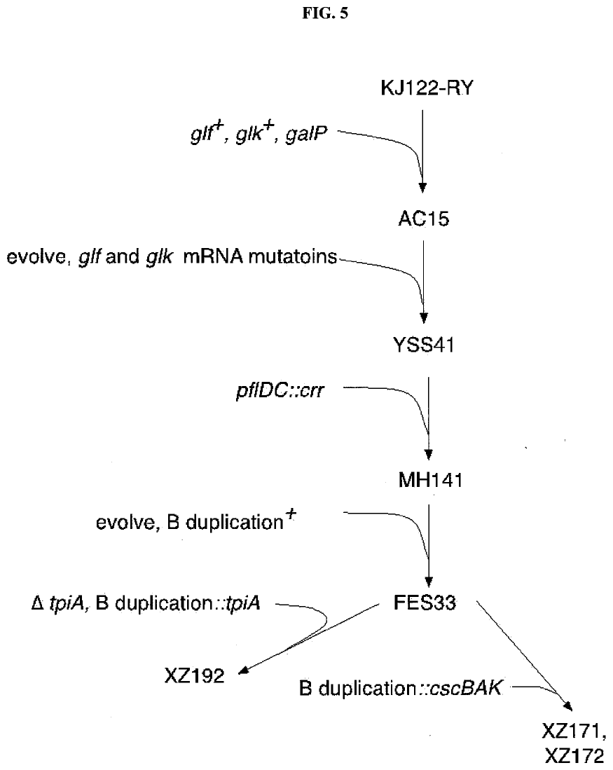

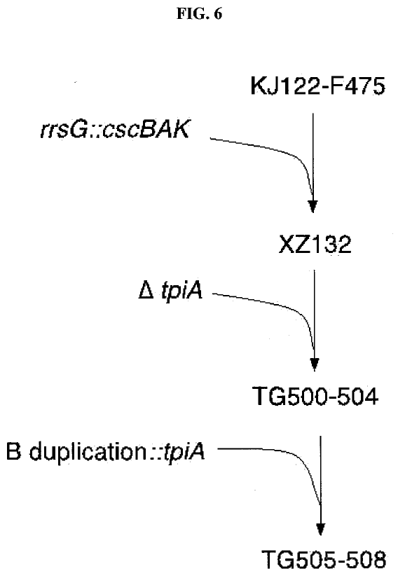

The construction of various strains in which the B duplication has been stabilized as described above in Example 1 is shown in FIGS. 5 and 6. All of the stabilized strains were constructed by using known methods of integrative transformation with linear DNA assisted by the lambda red recombinase system, followed by selection for homologous integration, followed by diagnostic PCR for correct integration, and/or by metabolic evolution (Jantama et al., 2008a; Jantama et al. 2008b).

### Example 5

### Stabilization of the B Duplication

Eighteen individual colonies of train XZ174, which contains the B duplication and the selectable marker cscBAK installed at the junction of the B duplication, and 13 individual colonies of strain XZ132, which contains the B duplication but does not contain the stabilizing selectable marker installed at the junction of the B duplication, were grown aerobically in liquid cultures containing sucrose as a carbon source, for about 50 generations. Each culture was then tested for presence of the B duplication by diagnostic PCR using primers BY296 and BY297, as described above, except that the elongation time at 68° C. was increased from two minutes to six minutes to accommodate the extra four kilobases of DNA comprising the cscBAK operon between the priming sites. All 18 cultures from the stabilized strain XZ174 retained the B duplication and selectable marker, giving a 5.8 kilobase PCR product, while for the strain XZ132, in which the B duplication has not been stabilized, two out of 13 cultures lost the B duplication. Thus, the selectable marker at the junction between the two copies of the B duplication did stabilize the copy number of the B duplication.

### Example 6

### Sucrose Fermentation by Different Escherichia coli Strains

Three different Escherichia coli strains described in this patent application were grown in 7 liter fermentation vessels using sucrose as the sole source of carbon in a minimal medium and their fermentation performance was monitored. Details of the fermentation methods for these fermentations as well those given in Table 1 were previously described in U.S. Pat. No. 9,845,513. The result from this fermentation study is provided in Table 5. The numbers shown are the average of two independent fermentations. Both of the two strains that contain the B duplication (XZ132 and XZ174) performed better than the control strain SD14, which does not contain the B duplication, with respect to titer, yield, and time of fermentation.

### Example 7

### Duplications that Lead to More than Two Copies

In some cases, duplication of a DNA sequence can lead to more than two copies of a DNA sequence in tandem (Fierro, Barredo et al. 1995). The number of copies can be determined by any of a number of appropriate methods, for example examining the read frequency in raw Illumina platform data, direct observation from raw PacBio data (if the duplication is not large), quantitative PCR, restriction digestion and agarose gel electrophoresis, or electrophoresis of whole chromosomes (for relatively large duplications). When there are n copies of a duplication, stabilization of all n copies can be achieved by installing a selectable maker between each adjacent pair of duplications as described in Example 1 above. It is preferred that a different selectable marker (that is a marker that can be selected for independently from any other marker used previously) be used for each adjacent pair of duplications, such that n copies of a duplication can be preferably stabilized with n−1 selectable markers. Since many different essential genes can be made to be conditionally required, there are many different possible selectable markers available, so many copies of a duplication can be stabilized. For example, the pyrF gene can be inactivated by mutation (preferably deleted) from an E. coli strain that contains a duplication that is desired to be stabilized, rendering the strain dependent on added uracil for growth. In a second step, installation of a functional pyrF gene (either native or heterologous homolog) at the junction of an adjacent pair of duplicated sequences will then stabilize that pair of duplications when the strain is grown in the absence of uracil. Thus, for example, if a duplication contains three copies of the duplicated DNA in an E. coli strain, a tipA gene can be used as the selectable marker for the first pair and a pyrF gene can be usd for the second pair. Given the large number of biosynthetic pathway genes in most microorganism, a large number of duplicated copies can be stabilized by extension and reiteration of this concept. Similarly, the TPI, URA3, and other biosynthetic yeast genes can be used in the same way in yeasts. In theory, any gene, for which a simple auxotrophy can found or constructed, can be exploited in this way.

### Example 8

### Further Enhancement of the B Duplication

One possible mechanism to explain the increase in succinate titer that results from the B duplication is that the B duplication results in a second copy of the first four genes of the menaquinone biosynthetic operon, menFDHB, and that a resulting increase in expression of those genes increases the concentration of menaquinone, which in turn increases the cells ability to grow under anaerobic or microaerobic conditions. However, since the IS4 element inserted in the middle of the menC gene, the last two genes of the men operon, menCE, are not duplicated in the B duplication. As such, insertion of an intact copy of menCE at the 3′ end of the first copy of the B duplication as drawn in FIG. 4, to give a complete second copy of the men operon, would further increase the capacity of the cell to synthesize menaquinone. Such an insertion can be easily accomplished by methods well known in the art for integrating linear DNA fragments into a chromosome by homologous recombination, for example integration of a cat, sacB cassette at the border between menC and the IS4 at the B duplication junction in a first step, selecting for chloramphenicol resistance, and then, in a second step, integration of a reconstituted menCE gene cassette by counterselecting against the sacB gene on plates containing sucrose (Jantama et al, 2008a; Jantama et al, 2008b; (Zhu, Tan et al. 2014); and U.S. Pat. No. 8,691,539). The reconstituted menCE cassette would contain appropriate flanking homology, for example about 500 base pairs upstream the 5′ end of the menC gene, and about 500 base pairs of the 5′ end of IS4.

### Example 9

### Stabilization of a Duplication in a Yeast

From the genome sequence of Kluyveromyces marxianus, it can be seen that the gene annotated as PDR12 exists as a duplication. From studies in Saccharomyces cerevisiae, Pdr12p is known to be a weak-acid-inducible multidrug transporter required for weak organic acid resistance. Deletion of PDR12 in a Saccharomyces cerevisiae strain led to increased sensitivity to inhibition by lactic acid (Nygard, Mojzita et al. 2014). As such, it can be deduced that duplication of the PDR12 homolog in K. marxianus could be important for its exceptional resistance to organic acids at low pH (Patent application WO 2014/043591). In order to prevent the loss of the PDR12 duplication, it is therefore logical to stabilize the PDR12 duplication in strains that are engineered to produce an organic acid by fermentation of a yeast. This can be done, similarly to the example give above, by inserting a selectable marker at the PsiI restriction site that is about 380 base pairs downstream from the stop codon of the first copy of the PDR12 gene. At all steps, the correct structure is confirmed by diagnostic PCR. For the first step, the URA3 coding sequence is deleted from an ancestor K. marxianus strain that contains the PDR12 duplication, such as strain DMKU3, by transforming with a linear deletion cassette (SEQ ID NO 11), and selecting for resistance to 5-fluoro orotic acid as described above. In the second step, a copy of the K. marxainus TPI gene is inserted at the duplication junction of the PRD12 duplication as follows. A second cassette (for example, SEQ ID NO 12) is assembled from the following DNA sequences in the order listed: (1) an upstream flanking homology comprising a copy of the 380 base pair sequence from the stop codon of the 5′ copy of the PDR12 gene to a PsiI restriction site, (2) a copy of the TPI gene including its promoter and terminator, (3) a copy of any 300 base pair DNA sequence that encodes no significant function and contains no homology to any sequence in the host strain (“sequence X”), (4) a copy of a Saccharomyces cerevisiae URA3 gene, including its promoter and terminator, (5) a second parallel copy of sequence X, and (6) a copy of the 400 base pair sequence just 3′ to the PsiI restriction site downstream form the 5′ copy of the PRD12 gene, described above. This cassette is transformed and integrated by selecting on a minimal glucose medium that lacks uracil. In a third step, the resulting strain is plated at about 10⁸ cells per plate containing 1 g/L 5-fluoroorotic acid and 24 mg uracil per liter, to counterselect against the URA3 gene, causing it to cross out by recombination between the two parallel copies of sequence X. In a fourth step, the TPI gene at its native locus is deleted as follows. A cassette is constructed comprising the following DNA sequences in the order listed (SEQ ID NO 13): (1) a copy of the 500 base pairs natively just upstream of the TPI1 promoter, without any overlap to the promoter carried with the TPI1 gene used in the second step described above, (2) a copy of a Saccharomyves cerevisiae URA3 gene including its promoter and terminator, (3) a copy of the 500 base pairs natively just downstream from the TPI1 terminator used in step 2 above. This cassette is then integrated into the strain from the third step 4 by selecting on plates containing a minimal glucose medium that lacks uracil. The resulting strain will contain the TPI1 gene, including its promoter and terminator, transplanted from its native locus to the junction between the two copies of the PDR12 gene. The native copy of the TPI1 gene will have been replaced with a Saccharomyces cerevisiae URA3 gene. A person skilled in the art will know that the above construction is just one example of how to stabilize the PDR12 duplication, and that many other functionally equivalent approaches are possible.

### Example 10

### Generalization of the Invention

Although the examples disclosed herein used E. coli or K. marxianus as the host organism and succinic acid as the desire product, those skilled in the art will know that homologous recombination and unequal crossover are well known phenomena in virtually all microorganisms where it has been examined. Thus, the methods and principles disclosed herein can be applied to many microbes and many DNA sequences, the only limitation being that here be a method for introducing exogenous DNA into the microorganism, for example by transformation, transduction, transfection, or mating. For example, Zygosaccharomyces bailii, a yeast well known for its tolerance to organic acids at low pH, contains three copies in tandem of a gene that is highly homologous to PDR12. These three tandemly duplicated genes are named BN860_04456g, BN860_04478g, and BN860_04500g in GenBank accession number HG316458, which discloses scaffold 5 of the genome sequence of Z. bailii strain CUB 213. The three copies can be stabilized by installing a selectable marker between each pair, as described herein.

The specific example given above involves a strain of E. coli that has been engineered to produce succinic acid, and which was improved by the duplication of a functional DNA sequence named the B duplication. However, the principles disclosed herein can be applied to any microbe where a tandem duplication of a functional DNA sequence can be generated by screening, selection, or metabolic evolution, and found by diagnostic PCR, DNA sequencing, gel electrophoresis, or a functional assay (for example an enzyme assay of an encoded gene, or titer of a product from fermentation). Once found, such a duplication can be stabilized to maintain its amplified copy number as disclosed herein by installing a suitable selectable marker between adjacent copies of the duplicated sequence. This can be accomplished in any microbe where a DNA sequence, comprising a selectable marker and flanking sequences homologous to (preferably identical to) sequences surrounding the junction between the duplicated sequence, can be installed, for example by transformation and homologous recombination between said flanking sequences and the sequences surrounding the duplication junction, that results in integration of the selectable marker at the junction between an adjacent pair of the duplicated sequence. In microbes where non-homologous end joining of incoming DNA is more frequent than homology-directed integration upon transformation with a selectable marker, it is useful to first mutate (preferably delete) one or more genes that are responsible for said non-homologous end joining, for example one or more of the NEJ1, KU70, KU80, or LIG4 genes in Kluveromyces marxianus. In some microbes, such as E. coli, where chromosomal integration upon transformation with a selectable marker is a relatively rare event, it is useful to first supply a function that increases the frequency of homologous recombination, for example the red genes from bacteriophage lambda, as contained on pKD46 or pCP225 (see Table 3, Jantama et al., 2008a, and Jantama et al., 2008b). Preferred microbes for applying this invention are those that are known to be useful for commercial purposes, such as microbes chosen from the genera Escherichia, Klebsiella, Saccharomyces, Penicillium, Bacillus, Issatchenkia, Pichia, Candida, Corynebacterium, Streptomyces, Actinomyces, Clostridium, Aspergillus, Trichoderma, Rhizopus, Mucor, Lactobacillus, Zygosaccharomyces, or Kluyveromyes.

TABLE 1
Genome structure of various KJ122 stocks
and phage resistant derivatives

| | A | B | Succiate |
|---|---|---|---|
| Strain | duplication | duplication | titer (g/L) |
| KJ122-F475 Stock | + | + | 89 |
| MYR592 MLPI, | + | — | 68 |
| LA1 phage^{R} | | | |
| MYR593 MLPI, | + | — | 49 |
| LA1 phage^{R} | | | |
| MYR585 MLPI, | — | — | 58 |
| LA1 phage^{R} | | | |
| LU16 Leuna and | — | + | 88 |
| LA1 phage^{R} | | | |
| MYR585-4E | — | + | 87 |
| phage^{R} evolved | | | |
| KJ122 - RY Stock | — | — | 57 |

TABLE 2
Bacterial strains used in the invention

| Bacterial | |
|---|---|
| strain | Genotype/Description |
| KJ122-F475 | E. coli Crooks, ΔldhA, ΔadhE, ΔackA, ΔfocA-pflB, |
| | ΔmgsA, ΔpoxB, ΔtdcDE, ΔcitF, ΔaspC, ΔsfcA, |
| | B duplication⁺(B+) |
| KJ122-RY | E. coli Crooks, ΔldhA, ΔadhE, ΔackA, ΔfocA-pflB, |
| | ΔmgsA, ΔpoxB, ΔtdcDE, ΔcitF, ΔaspC, ΔsfcA |
| AC15 | KJ122-RY, ΔptsHI, ΔgalP, glf⁺-glk⁺ |
| | (from Zymomonas mobilis) |
| YSS41 | AC15 evolved (two glf⁺-glk⁺ mRNA mutations) |
| MH141 | YSS41 pflD::crr |
| FES33 | MH141 evolved, B+ |
| XZ132 | KJ122-F475 rrsG::cscBAK |
| XZ156 | FES33 rrsG::cscBAK, B+ |
| XZ157 | |
| XZ158 | |
| XZ159 | |
| XZ162 | FES33 B+, pCP225 |
| XZ167 | FES33 ΔtpiA, B+ |
| XZ171 | FES33 B+::cscBAK |
| XZ172 | |
| XZ173 | KJ122-F475 B+::cscBAK |
| XZ174 | |
| XZ175 | XZ167 pKD46, Amp^{R} |
| XZ192 | FES33 ΔtpiA, B+::tpiA |
| TG500 | XZ132 ΔtpiA |
| TG501 | |
| TG502 | |
| TG503 | |
| TG504 | |
| TG505 | TG500 ΔtpiA, B+::tpiA |
| TG506 | |
| TG507 | |
| TG508 | |

TABLE 3
Plasmids used in the invention

| Plasmid | Genotype/Description |
|---|---|
| pKD46 | P_{BAD} redα redβ redγ, amp^{R} |
| pCP225 | pKD46 + cas9, amp^{R} kan^{R} |
| pXZ016 | A plasmid for carrying gRNA for the tpiA deletion |
| pXZ017 | pCL1921 cscBAK, spc^{R}, a plasmid built for |
| | integrating the sucrose utilization gene cassette cscBAK |
| | at the B duplication junction site, with two flanking |
| | homologies of about 450 basepairs each. |
| pXZ020 | pBluescript II KS-tpiA, amp^{R}, a plasmid |
| | built for integrating the tpiA gene at the B duplication |
| | junction site, with 457bp of upstream and 447bp of |
| | downstream flanking homologies. |

TABLE 4
Sequence information

| No. | Name | Description |
|---|---|---|
| 1 | SEQ ID NO 1 | Zymomonas mobilis glf open reading frame (orf) nucleotide |
| | | sequence |
| 2 | SEQ ID NO 2 | Zymomonas mobilis glk open reading frame (orf) nucleotide |
| | | sequence |
| 3 | SEQ ID NO 3 | E. coli W (ATCC 9637) cscB open reading frame (orf) encoding |
| | | sucrose permease-proton symporter, nucleotide sequence |
| 4 | SEQ ID NO 4 | E. coli W (ATCC 9637) cscA open reading frame (orf) encoding |
| | | invertase nucleotide sequence |
| 5 | SEQ ID NO 5 | E. coli W (ATCC 9637) cscK open reading frame (orf) encoding |
| | | fructose-6 kinase, nucleotide sequence |
| 6 | SEQ ID NO 6 | E. coli Crooks tpiA open reading frame (orf) encoding |
| | | triosephosphate isomerase nucleotide sequence |
| 7 | SEQ ID NO 7 | E. coli Crooks tpiA deletion sequence |
| 8 | SEQ ID NO 8 | B Duplication junction site nucleotide sequence; duplication starts at |
| | | the 139 nt of the EcolC_1387 gene of the E. coli genome |
| 9 | SEQ ID NO 9 | E. coli Crooks TG505 tpiA insertion into junction site of the B |
| | | Duplication |
| 10 | SEQ ID NO 10 | E. coli Crooks XZ13 or XZ174 cscBAK insertion into junction site |
| | | of the B Duplication |
| 11 | SEQ ID NO 11 | K. marxianus URA3 deletion cassette |
| 12 | SEQ ID NO 12 | K. marxianus cassette for stabilizing the PDR12 duplication |
| 13 | SEQ ID NO 13 | Cassette for replacing the TPI1 gene in K. marxianus with a |
| | | S. cerevisiae URA3 gene |
| 14 | SEQ ID NO 14 | Primer for the cscBAK cassette at the rrsG site from SD14 |
| 15 | SEQ ID NO 15 | Primer for the cscBAK cassette at the rrsG site from SD14 |
| 16 | SEQ ID NO 16 | Primer for making a gRNA for creating a tpiA deletion located in the |
| | | middle of the ORF of the tpiA gene |
| 17 | SEQ ID NO 17 | Synthetic gBlock fragment for constructing the tpiA deletion on |
| | | chromosome |
| 18 | SEQ ID NO 18 | Diagnostic primer for the tpiA deletion |
| 19 | SEQ ID NO 19 | Diagnostic primer for the tpiA deletion |
| 20 | SEQ ID NO 20 | Primer for the cscBAK cassette (B:cscAKB, 4163 bp) from SD14 |
| 21 | SEQ ID NO 21 | Primer for the cscBAK cassette (B:cscAKB, 4163 bp) from SD14 |
| 22 | SEQ ID NO 22 | Primer for the cscBAK cassette (B:cscAKB, 4123 bp) from SD14 |
| 23 | SEQ ID NO 23 | Primer for the cscBAK cassette (B:cscAKB, 4123 bp) from SD14 |
| 24 | SEQ ID NO 24 | Primer for the backbone of plasmid pCL1921 |
| 25 | SEQ ID NO 25 | Primer for the backbone of plasmid pCL1921 |
| 26 | SEQ ID NO 26 | Synthetic gBlock fragment to be used as the upstream homology for |
| | | cloning the cscBAK cassette at the B duplication junction site |
| 27 | SEQ ID NO 27 | Synthetic gBlock fragment to be used as the downstream homology |
| | | for cloning the cscBAK cassette at the B duplication junction site |
| 28 | SEQ ID NO 28 | Primer for cloning the cscBAK cassette at the B duplication site from |
| | | pXZ017 |
| 29 | SEQ ID NO 29 | Primer for cloning the cscBAK cassette at the B duplication site from |
| | | pXZ017 |
| 30 | SEQ ID NO 30 | Primer for cloning the tpiA fragment (1161 bp) at the B duplication |
| | | site from KJ122 gDNA |
| 31 | SEQ ID NO 31 | Primer for cloning the tpiA fragment (1161 bp) at the B duplication |
| | | site from KJ122 gDNA |
| 32 | SEQ ID NO 32 | Primer for the backbone of the pBluescript II KS(−) plasmid for the |
| | | construction of pXZ020 |
| 33 | SEQ ID NO 33 | Primer for the backbone of the pBluescript II KS(−) plasmid for the |
| | | construction of pXZ020 |
| 34 | SEQ ID NO 34 | Synthetic gBlock fragmeng to be used as the upstream homology for |
| | | cloning the tpiA fragment at the B duplication junction site |
| 35 | SEQ ID NO 35 | Synthetic gBlock fragmeng to be used as the downstream homology |
| | | for cloning the tpiA fragment at the B duplication junction site |
| 36 | SEQ ID NO 36 | Primer the B::tpiA fragment from plasmid pXZ020 |
| 37 | SEQ ID NO 37 | Diagnostic primer for the B duplication with BY297 |
| 38 | SEQ ID NO 38 | Diagnostic primer for the B duplication with BY296 |

TABLE 5
Fermentation of sucrose to succinic acid by various strains

| | | B | | | Average | Elapsed |
|---|---|---|---|---|---|---|
| | B | Dupli- | Average | Average | Succincic | Fermen- |
| | Dupli- | cation | Succinic | Acetic | Acid | tation |
| | cation | Stabilized | Acid | Acid | Yield | Time |
| Strain | Present | By: | (g/L) | (g/L) | (g/g) | (hr) |
| XZ132 | + | — | 80 | 6.7 | 0.89 | 36 |
| XZ174 | + | cscBAK | 80 | 5.0 | 0.88 | 36 |
| SD14 | − | — | 74 | 5.1 | 0.83 | 45 |

### REFERENCES

European Patent No. 1214420
U.S. Pat. No. 7,527,927
U.S. Pat. No. 8,691,539
U.S. Pat. No. 8,871,489
International Patent App. Pub. No. WO 2011/063055
International Patent App. Pub. No. WO 2011/373671
International Patent App. Pub. No. WO 2012/082720
International Patent App. Pub. No. WO 2015/013334
Altschul, S. F., W. Gish, W. Miller, E. W. Myers and D. J. Lipman (1990). “Basic local alignment search tool.” J Mol Biol 215(3): 403-410.
Altschul, S. F., T. L. Madden, A. A. Schaffer, J. Zhang, Z. Zhang, W. Miller and D. J. Lipman (1997). “Gapped BLAST and PSI-BLAST: a new generation of protein database search programs.” Nucleic Acids Res 25(17): 3389-3402.
Elliott, K. T., L. E. Cuff and E. L. Neidle (2013). “Copy number change: evolving views on gene amplification.” Future Microbiol 8(7): 887-899.
Fierro, F., J. L. Barredo, B. Diez, S. Gutierrez, F. J. Fernandez and J. F. Martin (1995). “The penicillin gene cluster is amplified in tandem repeats linked by conserved hexanucleotide sequences.” Proc Natl Acad Sci USA 92(13): 6200-6204.
Guest, J. R. (1977). “Menaquinone biosynthesis: mutants of Escherichia coli K-12 requiring 2-succinylbenzoate.” J Bacteriol 130(3): 1038-1046. Jantama, K., M. J. Haupt, S. A. Svoronos, X. Zhang, J. C. Moore, K. T. Shanmugam and L. O.
Ingram (2008a). “Combining metabolic engineering and metabolic evolution to develop nonrecombinant strains of Escherichia coli C that produce succinate and malate.” Biotechnol Bioeng 99(5): 1140-1153.
Jantama, K., X. Zhang, J. C. Moore, K. T. Shanmugam, S. A. Svoronos and L. O. Ingram (2008b). “Eliminating side products and increasing succinate yields in engineered strains of Escherichia coli C.” Biotechnol Bioeng 101(5): 881-893.
Lopes, T. S., I. J. de Wijs, S. I. Steenhauer, J. Verbakel and R. J. Planta (1996). “Factors affecting the mitotic stability of high-copy-number integration into the ribosomal DNA of Saccharomyces cerevisiae.” Yeast 12(5): 467-477.
Nygard, Y., D. Mojzita, M. Toivari, M. Penttila, M. G. Wiebe and L. Ruohonen (2014). “The diverse role of Pdr12 in resistance to weak organic acids.” Yeast 31(6): 219-232.
Reams, A. B. and J. R. Roth (2015). “Mechanisms of gene duplication and amplification.” Cold Spring Harb Perspect Biol 7(2): a016592.
Tyo, K. E., P. K. Ajikumar and G. Stephanopoulos (2009). “Stabilized gene duplication enables long-term selection-free heterologous pathway expression.” Nat Biotechnol 27(8): 760-765.
Zhu, X., Z. Tan, H. Xu, J. Chen, J. Tang and X. Zhang (2014). “Metabolic evolution of two reducing equivalent-conserving pathways for high-yield succinate production in Escherichia coli.” Metab Eng 24: 87-96.

What is claimed is:

1. A non-naturally occurring microorganism with a stabilized copy number of a functional DNA sequence, comprising n copies of a functional DNA sequence and at least one selectable marker between at least one adjacent pair of said n copies of said functional DNA sequence, wherein said non-naturally occurring microorganism is a descendent of an ancestor microorganism, wherein said ancestor microorganism comprises no more than n−1 copies of said functional DNA sequence, and wherein n is at least 2.

2. The non-naturally occurring microorganism of claim 1, wherein said microorganism is a bacterium, yeast, filamentous fungus, or archaea.

3. The non-naturally occurring microorganism of claim 1, wherein said microorganism is selected from the group consisting of Escherichia, Klebsiella, Saccharomyces, Penicillium, Bacillus, Issatchenkia, Pichia, Candida, Corynebacterium, Streptomyces, Actinomyces, Clostridium, Aspergillus, Trichoderma, Rhizopus, Mucor, Lactobacillus, Zygosaccharomyces, and Kluyveromyes.

4. The non-naturally occurring microorganism of claim 1, wherein said microorganism is an Escherichia coli or Kluyveromyces marxianus.

5. The non-naturally occurring microorganism of claim 4, wherein said functional DNA sequence is the sequence duplicated in the B duplication, a PDR12 gene, or a homolog of a PDR12 gene.

6. The non-naturally occurring microorganism of claim 1, wherein said selectable marker is a gene encoding triosephosphate isomerase or a cassette for metabolizing sucrose.

7. The non-naturally occurring microorganism of claim 1, wherein said microorganism produces a titer of a desired fermentation product that is at least 10 percent greater than the titer of said desired fermentation product produced by said ancestor microorganism under similar culture conditions.

8. A method for creating a non-naturally occurring microorganism with a stabilized copy number of a functional DNA sequence, comprising the steps of:
(a) providing a non-naturally occurring microorganism comprising n copies of a functional DNA sequence, wherein said non-naturally occurring microorganism is a descendent of an ancestor microorganism, wherein said ancestor microorganism comprises no more than n−1 copies of said functional DNA sequence, and wherein n is at least 2; and
(b) inserting into said non-naturally occurring microorganism at least one selectable marker between at least one adjacent pair of said n copies of said functional DNA sequence to stabilize the copy number of said functional DNA sequence.

9. The method of claim 8, wherein said microorganism is a bacterium, yeast, filamentous fungus, or archaea.

10. The method of claim 9, wherein said microorganism is selected from the group consisting of Escherichia, Klebsiella, Saccharomyces, Penicillium, Bacillus, Issatchenkia, Pichia, Candida, Corynebacterium, Streptomyces, Actinomyces, Clostridium, Aspergillus, Trichoderma, Rhizopus, Mucor, Lactobacillus, Zygosaccharomyces, and Kluyveromyes.

11. The method of claim 8, wherein said microorganism is an Escherichia coli or a Kluyveromyces marxianus.

12. The method of claim 11, wherein said functional DNA sequence is the sequence duplicated in the B duplication, a PDR12 gene, or a homolog of a PDR12 gene.

13. A method for identifying a descendant microorganism having at least one improved fermentation parameter resulting from a duplication of a functional DNA sequence of an ancestor microorganism, comprising the steps of:
(a) growing said ancestor micoorganism under a first set of fermentation conditions that generates at least one descendant microorganism;
(b) growing said at least one descendant microorganism under a second set of fermentation conditions;
(c) identifying at least one descendant microorganism having at least one improved fermentation parameter as compared to said ancestor microorganism;
(d) determining a DNA sequence of said at least one descendant microorganism having at least one improved fermentation parameter and the DNA sequence of said ancestor microorganism; and
(e) comparing said DNA sequence of said at least one descendant microorganism having at least one improved fermentation parameter to the DNA sequence of said ancestor microorganism to identify a duplication of said functional DNA sequence in said descendant microorganism.

14. The method of claim 13, wherein said first set of fermentation conditions is anaerobic or microaerobic growth and said second set of fermentation conditions is aerobic growth.

15. The method of claim 13, wherein said first set of fermentation conditions is aerobic growth and said second set of fermentation conditions is anaerobic or microaerobic growth.

16. The method of claim 13, wherein said ancestor microorganism is a bacterium, yeast, filamentous fungus, or archaea.

17. The method of claim 13, wherein said ancestor microorganism is selected from the group consisting of Escherichia, Klebsiella, Saccharomyces, Penicillium, Bacillus, Issatchenkia, Pichia, Candida, Corynebacterium, Streptomyces, Actinomyces, Clostridium, Aspergillus, Trichoderma, Rhizopus, Mucor, Lactobacillus, Zygosaccharomyces, and Kluyveromyes.

18. The method of claim 13, wherein said microorganism is an Escherichia coli or a Kluyveromyces marxianus.

19. The method of claim 13, wherein said functional DNA sequence is the sequence duplicated in the B duplication, a PDR12 gene, or a homolog of a PDR12 gene.
